(19) United States
(12) Patent Application Publication
(10) Pub. No.: US 2025/0215032 A1
(43) Pub. Date: Jul. 3, 2025

(54) PHOSPHORYLPURINONE COMPOUNDS FOR THE TREATMENT OF CANCER

(71) Applicant: Hoffmann-La Roche Inc., Little Falls, NJ (US)

(72) Inventors: Chungen LIANG, Shanghai (CN); Jianping WANG, Shanghai (CN); Junjie WANG, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 19/010,516

(22) Filed: Jan. 6, 2025

Related U.S. Application Data
(63) Continuation of application No. PCT/EP2023/069250, filed on Jul. 12, 2023.

(30) Foreign Application Priority Data
Jul. 14, 2022 (WO) PCT/CN2022/105719

(51) Int. Cl. C07F 9/6561; A61K 31/675

(52) U.S. Cl. CPC C07F 9/65616; A61K 31/675

(57) ABSTRACT

The present invention relates to compounds of formula (I),

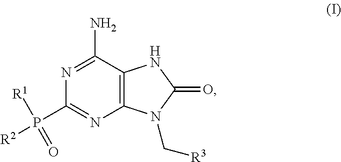

wherein R¹ to R³ are as described herein, and their pharmaceutically acceptable salt thereof, and compositions including the compounds and methods of using the compounds.

The present invention relates to organic compounds useful for therapy and/or prophylaxis in a mammal, and in particular to agonism of TLR7 useful for treating cancers.

### FIELD OF THE INVENTION

The present invention relates to novel phosphorylpurinones and their derivatives that have Toll-like receptor agonism activity thereof, as well as their manufacture, pharmaceutical compositions containing them and their potential use as medicaments for the treatment and/or prophylaxis of cancers.

Although immune checkpoint inhibitors have changed the treatment landscape of many tumors, durable responses are limited. The lymphocyte infiltration and IFN-γ status are key factors for effective therapy by defining a “T cell inflamed” phenotype (“hot tumors”). In contrast, lack of T cells infiltrating the tumor characterizes “non-inflamed” or “cold tumors”. Immunological treatment of cold tumors is a great challenge as no adaptive immune response has been set up or maintained. Cold tumors can however contain substantial numbers of myeloid cells including macrophages, different subsets of DCs and myeloid derived suppressor cells. Agonistic approaches targeting myeloid cells aim to induce anti-tumor inflammatory responses by secretion of pro-inflammatory cytokines and in turn supporting T cell effector function and memory formation. Given the presence of myeloid cells also in cold tumors, activating them in the tumor microenvironment will increase T cell infiltration and sustained activation in these tumors. This can translate into clinical benefit in inflamed and immune excluded tumors, where only a fraction of the patients receives benefit from immunotherapy to date.

As a part of the innate immune system, myeloid cells are capable of reacting to infection and aberrant cell behavior. They are the first to detect the pathogen/aberrant cells and eliminate them by different mechanisms including inflammatory cytokine secretion and phagocytosis. Sustained activation of T cells by providing co-stimulatory signals and antigen presentation to T cells is another pivotal function of myeloid cells paving way to a long lasting immune response. On the other hand, they also play a key role in tissue repair and homeostasis by suppressing uncontrolled immune activation. Tumors take advantage of this property of these cells by recruiting them where they create a suppressive microenvironment thereby inhibiting T cells by direct and indirect mechanisms. Tumor associated myeloid cells can still be activated in the suppressive tumor microenvironment by delivering the right signals for e.g. TLR agonists. Upon activation, they can produce inflammatory cytokines and upregulate activation markers and thus trigger an anti-tumor immune response, ideally resulting in long lasting adaptive response.

Toll-like receptors (TLR) detect highly conserved, pathogenic danger signals resulting in a strong inflammatory response. TLR signaling provokes an innate and ultimately an adaptive immune response against the pathogen. Toll-like receptors (TLRs) detect a wide range of conserved pathogen-associated molecular patterns (PAMPs). They play an important role in sensing invading pathogens and subsequent initiation of innate immune responses. There are 10 known members of the TLR family in humans, which are type I transmembrane proteins featuring an extracellular leucine-rich domain and a cytoplasmic tail that contains a conserved Toll/interleukin (IL)-1 receptor (TIR) domain. Within this family, TLR3, TLR7, TLR8 and TLR9 are located within endosomes.

TLR7 can be activated by binding to a specific small molecule ligand (i.e., TLR7 agonist) or its native ligand (i.e., single-stranded RNA, ssRNA). Following binding of ssRNA to TLR7, the receptor in its dimerized form is believed to undergo a structural change leading to the subsequent recruitment of adapter proteins at its cytoplasmic domain, including the myeloid differentiation primary response gene 88 (MyD88). Following the initiation of the receptor signaling cascade via the MyD88 pathway, cytoplasmic transcription factors such as interferon regulatory factor 7 (IRF-7) and nuclear factor kappa B (NF-κB) are activated. These transcription factors then translocate to the nucleus and initiate the transcription of various genes, e.g., IFN-α and other antiviral cytokine genes. Of high interest is the activation of intra-tumoral myeloid cells that initiate and orchestrate immune responses and express TLR7 in the endosome (natural ligand is single stranded RNA from viruses). Systemic administration of TLR agonists leads to strong systemic activation of different immune cells and eventually immune cells within the tumor microenvironment are activated to achieve anti-tumor effect. In fact, several identified TLR7 agonists have been considered for therapeutic purposes. Topical application of the TLR7 agonist Imiquimod (R-837) onto murine subcutaneous tumors or human patients with cutaneous cancer lesions (Aldara®) strongly activates an anti-tumor immune response. The TLR7/8 dual agonist Resiquimod (R-848) is an immune modulating topical gel in phase II clinical trials at Galderma for the treatment of stage cutaneous T-cell lymphoma. MBS-8 is a micellar nanoparticle formulation of the TLR7 agonist 1V270 being developed by MonTa Biosciences as an intravenous immunotherapeutic treatment for advanced solid tumors. BDB-001 is a TLR 7/8 dual agonist in phase II clinical trials at Seven and Eight Biopharmaceuticals as an intravenous treatment for advanced or metastatic solid tumors refractory to anti-PD-1/anti-PD-L1 therapy. APR-003 is an oral small molecule TLR7 agonist in early clinical development at Apros Therapeutics for the treatment of patients with advanced unresectable colorectal cancer with malignant liver lesions. PRTX-007 is an orally administered, small molecule TLR7 agonist in early clinical development at Primmune Therapeutics for the treatment of acute viral diseases, including SARS-CoV-2, and cancer. As such, there remains a need to identify further compounds that are suitable for systemic administration and preferentially tumor targeting. There is high unmet clinical need for developing potent and safe TLR7 agonists for the treatment of a broad spectrum of cancers.

### SUMMARY OF THE INVENTION

The present invention relates to novel compounds of formula (I),

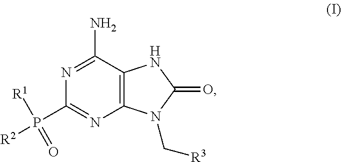

wherein
R¹ is C₁₋₆alkyl;
R² is C₁₋₆alkyl;
R³ is pyridinyl substituted by ((C₁₋₆alkyl)₂amino)C₁₋₆alkoxy, ((C₁₋₆alkyl)₂amino)C₁₋₆alkylamino, ((C₁₋₆alkyl)₂amino)C₁₋₆alkylpyrrolidinyl, (C₁₋₆alkyl)₂piperazinyl, (C₁₋₆alkylamino)C₁₋₆alkylamino, (C₁₋₆alkylpyrrolidinyl)amino, 2,3,3a,4,6,6a-hexahydro-1H-pyrrolo[3,4-c]pyrrolyl, 2,5-diazabicyclo[2.2.2]octanyl, aminopyrrolidinyl, C₁₋₆alkyl-1-oxa-4,9-diazaspiro[5.5]undecanyl, C₁₋₆alkylpiperazinyl, C₁₋₆alkylsulfonylpiperazinyl, pyrrolidinylC₁₋₆alkylamino or tetrahydropyranylamino;
or a pharmaceutically acceptable salt thereof.

The invention also relates to their manufacture, medicaments based on a compound in accordance with the invention and their production as well as the use of compounds of formula (I) thereof as agonist of TLR7.

The compounds of formula (I) show good TLR7 agonism activity. In another embodiment, the compounds of this invention showed superior kinetic solubility and thermodynamic (equilibrium) solubility compared with reference compound RO14. In addition, the compounds of formula (I) show much improved fasted state simulated intestinal fluid (FaSSIF) and fed state simulated intestinal fluid (FeSSIF) dissolution results compared to reference compound.

### BRIEF DESCRIPTION OF THE FIGURE

FIG. 1. X-ray crystallographic analysis of Example 15-B

FIG. 2. X-ray crystallographic analysis of Compound 18e-A

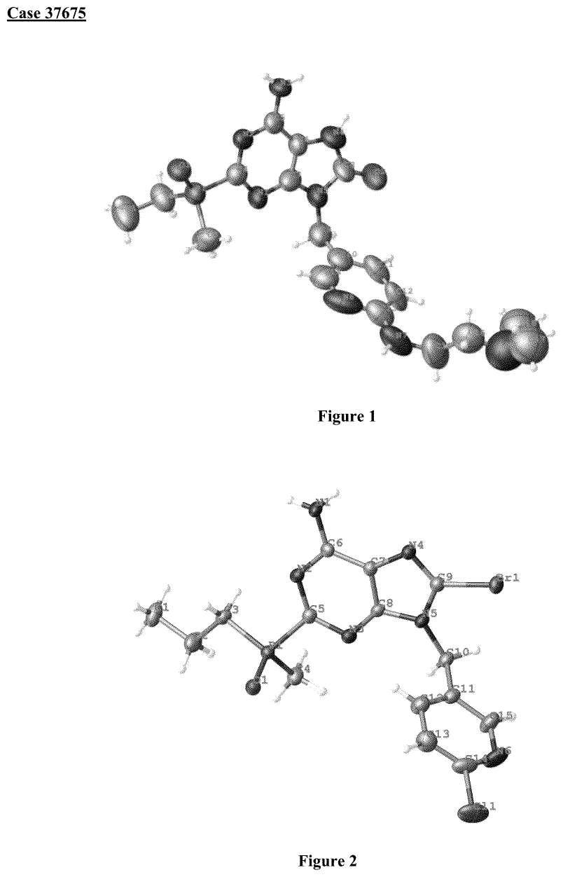

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkoxy” denotes C₁₋₆alkyl-O—.

The term “halogen” and “halo” are used interchangeably herein and denote fluoro, chloro, bromo, or iodo.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Agonist of TLR7

The present invention relates to (i) a compound of formula (I),

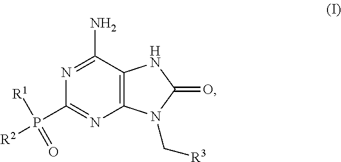

wherein
R¹ is C₁₋₆alkyl;
R² is C₁₋₆alkyl;
R³ is pyridinyl substituted by ((C₁₋₆alkyl)₂amino)C₁₋₆alkoxy, ((C₁₋₆alkyl)₂amino)C₁₋₆alkylamino, ((C₁₋₆alkyl)₂amino)C₁₋₆alkylpyrrolidinyl, (C₁₋₆alkyl)₂piperazinyl, (C₁₋₆alkylamino)C₁₋₆alkylamino, (C₁₋₆alkylpyrrolidinyl)amino, 2,3,3a,4,6,6a-hexahydro-1H-pyrrolo[3,4-c]pyrrolyl, 2,5-diazabicyclo[2.2.2]octanyl, aminopyrrolidinyl, C₁₋₆alkyl-1-oxa-4,9-diazaspiro[5.5]undecanyl, C₁₋₆alkylpiperazinyl, C₁₋₆alkylsulfonylpiperazinyl, pyrrolidinylC₁₋₆alkylamino or tetrahydropyranylamino;
or a pharmaceutically acceptable salt thereof.

A further embodiment of present invention is (ii) a compound of formula (I) according to (i), or a pharmaceutically acceptable salt thereof, wherein R¹ is methyl or ethyl.

A further embodiment of present invention is (iii) a compound of formula (I), according to (i) or (ii), or a pharmaceutically acceptable salt thereof, wherein R¹ is methyl.

A further embodiment of present invention is (iv) a compound of formula (I) according to any one of (i) to (iii), wherein R² is methyl, ethyl or propyl.

A further embodiment of present invention is (v) a compound of formula (I), or a pharmaceutically acceptable salt thereof, according to any one of (i) to (iv), wherein R³ is

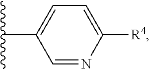

wherein R⁴ is ((C₁₋₆alkyl)₂amino)C₁₋₆alkoxy, ((C₁₋₆alkyl)₂amino)C₁₋₆alkylamino, ((C₁₋₆alkyl)₂amino)C₁₋₆alkylpyrrolidinyl, (C₁₋₆alkyl)₂piperazinyl, (C₁₋₆alkylamino)C₁₋₆alkylamino, (C₁₋₆alkylpyrrolidinyl)amino, 2,3,3a,4,6,6a-hexahydro-1H-pyrrolo[3,4-c]pyrrolyl, 2,5-diazabicyclo[2.2.2]octanyl, aminopyrrolidinyl, C₁₋₆alkyl-1-oxa-4,9-diazaspiro[5.5]undecanyl, C₁₋₆alkylpiperazinyl, C₁₋₆alkylsulfonylpiperazinyl, pyrrolidinylC₁₋₆alkylamino or tetrahydropyranylamino.

A further embodiment of present invention is (vi) a compound of formula (I), or a pharmaceutically acceptable salt thereof, according to any one of (i) to (v), wherein R³ is

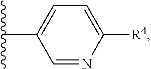

wherein R⁴ is ((C₁₋₆alkyl)₂amino)C₁₋₆alkylamino or (C₁₋₆alkylamino)C₁₋₆alkylamino.

A further embodiment of present invention is (vii) a compound of formula (I), or a pharmaceutically acceptable salt thereof, according to any one of (i) to (vi), wherein R³ is 2-(dimethylamino)ethylamino or 2-(methylamino)ethylamino.

A further embodiment of present invention is (viii) a compound of formula (I), according to any one of (i) to (vii), wherein
R¹ is C₁₋₆alkyl;
R² is C₁₋₆alkyl;
R³ is

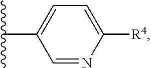

wherein R⁴ is ((C₁₋₆alkyl)₂amino)C₁₋₆alkylamino or (C₁₋₆alkylamino)C₁₋₆alkylamino;
or a pharmaceutically acceptable salt thereof.

A further embodiment of present invention is (ix) a compound of formula (I), according to any one of (i) to (viii), wherein
R¹ is methyl;
R² is methyl, ethyl or propyl;
R³ is

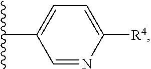

wherein R⁴ is 2-(dimethylamino)ethylamino or 2-(methylamino)ethylamino;
or a pharmaceutically acceptable salt thereof.

Another embodiment of present invention is a compound of formula (I) selected from the following:
6-amino-9-[[6-[2-(dimethylamino)ethylamino]-3-pyridyl]methyl]-2-dimethylphosphoryl-7H-purin-8-one;
6-amino-9-[[6-[2-(dimethylamino)ethoxy]-3-pyridyl]methyl]-2-dimethylphosphoryl-7H-purin-8-one;
6-amino-9-[[6-(3-aminopyrrolidin-1-yl)-3-pyridyl]methyl]-2-dimethylphosphoryl-7H-purin-8-one;
6-amino-2-dimethylphosphoryl-9-[[6-(3,3-dimethylpiperazin-1-yl)-3-pyridyl]methyl]-7H-purin-8-one;
6-amino-9-[[6-(2,5-diazabicyclo[2.2.2]octan-2-yl)-3-pyridyl]methyl]-2-dimethylphosphoryl-7H-purin-8-one;
9-[[6-(2,3,3a,4,6,6a-hexahydro-1H-pyrrolo[3,4-c]pyrrol-5-yl)-3-pyridyl]methyl]-6-amino-2-dimethylphosphoryl-7H-purin-8-one;
6-amino-2-dimethylphosphoryl-9-[[6-(2-pyrrolidin-1-ylethylamino)-3-pyridyl]methyl]-7H-purin-8-one;
6-amino-2-dimethylphosphoryl-9-[[6-(tetrahydropyran-4-ylamino)-3-pyridyl]methyl]-7H-purin-8-one;
6-amino-9-[[6-[3-[(dimethylamino)methyl]pyrrolidin-1-yl]-3-pyridyl]methyl]-2-dimethylphosphoryl-7H-purin-8-one;
6-amino-2-dimethylphosphoryl-9-[[6-(4-methyl-1-oxa-4,9-diazaspiro[5.5]undecan-9-yl)-3-pyridyl]methyl]-7H-purin-8-one;
6-amino-2-dimethylphosphoryl-9-[[6-[(1-methylpyrrolidin-3-yl)amino]-3-pyridyl]methyl]-7H-purin-8-one;
6-amino-2-dimethylphosphoryl-9-[[6-[2-[ethyl(methyl)amino]ethylamino]-3-pyridyl]methyl]-7H-purin-8-one;
6-amino-2-[(R)-ethyl(methyl)phosphoryl]-9-[[6-(4-methylpiperazin-1-yl)-3-pyridyl]methyl]-7H-purin-8-one;
6-amino-2-[(S)-ethyl(methyl)phosphoryl]-9-[[6-(4-methylpiperazin-1-yl)-3-pyridyl]methyl]-7H-purin-8-one;
6-amino-2-[(R)-ethyl(methyl)phosphoryl]-9-[[6-(4-methylsulfonylpiperazin-1-yl)-3-pyridyl]methyl]-7H-purin-8-one;
6-amino-2-[(S)-ethyl(methyl)phosphoryl]-9-[[6-(4-methylsulfonylpiperazin-1-yl)-3-pyridyl]methyl]-7H-purin-8-one;
6-amino-9-[[6-[2-(dimethylamino)ethylamino]-3-pyridyl]methyl]-2-[(R)-ethyl(methyl)phosphoryl]-7H-purin-8-one;
6-amino-9-[[6-[2-(dimethylamino)ethylamino]-3-pyridyl]methyl]-2-[(S)-ethyl(methyl)phosphoryl]-7H-purin-8-one;
6-amino-9-[[6-[2-(dimethylamino)ethoxy]-3-pyridyl]methyl]-2-[(R)-ethyl(methyl)phosphoryl]-7H-purin-8-one;
6-amino-9-[[6-[2-(dimethylamino)ethoxy]-3-pyridyl]methyl]-2-[(S)-ethyl(methyl)phosphoryl]-7H-purin-8-one;
6-amino-2-[(R)-ethyl(methyl)phosphoryl]-9-[[6-[2-(methylamino)ethylamino]-3-pyridyl]methyl]-7H-purin-8-one;
6-amino-9-[[6-[2-(dimethylamino)ethylamino]-3-pyridyl]methyl]-2-[(S)-methyl(propyl)phosphoryl]-7H-purin-8-one;
6-amino-9-[[6-[2-(dimethylamino)ethylamino]-3-pyridyl]methyl]-2-[(R)-methyl(propyl)phosphoryl]-7H-purin-8-one; and
6-amino-2-diethylphosphoryl-9-[[6-[2-(dimethylamino)ethylamino]-3-pyridyl]methyl]-7H-purin-8-one;
or a pharmaceutically acceptable salt thereof.

Another embodiment of present invention is related to (xi) a process for the preparation of a compound according to any one of (i) to (x) comprising the following step:
a) reaction between compound of formula (III),

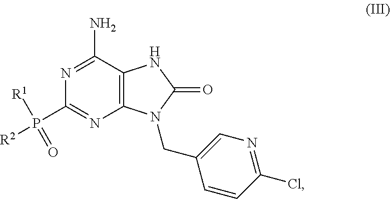

and an amine or alcohol HR⁴ (II), in a neat reaction; or in the presence of inorganic base, wherein the base is sodium hydride; or in the presence of organometallic catalyst system, wherein the catalyst system is selected from Pd₂(dba)₃/RuPhos/t-BuONa, Pd₂(dba)₃/BrettPhos/t-BuONa, and Pd-PEPPSI-IPentCl/t-BuOK; wherein R¹, R², and R⁴ are defined as in any one of (i) to (ix).

Another embodiment of present invention is (xii) a compound or pharmaceutically acceptable salt according to any one of (i) to (x), when manufactured according to the process of claim 11.

Another embodiment of present invention is (xiii) a pharmaceutical composition comprising a compound in accordance with any one of (i) to (x) and a pharmaceutically acceptable excipient.

Another embodiment of present invention is (xiv) a compound or pharmaceutically acceptable salt according to any one of (i) to (x) for use as therapeutically active substance.

Another embodiment of present invention is (xv) a compound or a pharmaceutically acceptable salt thereof according to any one of claims 1 to 10 for use in the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

Another embodiment of present invention is (xvi) the use of a compound according to any one of (i) to (x) as an agonist of TLR7.

Another embodiment of present invention is (xvii) the use of a compound according to any one of (i) to (x) for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

Another embodiment of present invention is (xviii) the use of a compound according to any one of (i) to (x) for the preparation of a medicament for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma.

Another embodiment of present invention is (xix) the use of a compound according to any one of (xvii) or (xviii), wherein the cancer is selected from pancreatic ductal adenocarcinoma and colorectal carcinoma.

Another embodiment of present invention is (xx) a method for the treatment of cancer, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma, which method comprises administering a therapeutically effective amount of a compound as defined in any one of (i) to (x).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to activate TLR7 receptor and lead to produce INF-α and other cytokines, which can be used, but not limited, for the treatment of cancers. For example, such amount may be below the amount that is toxic to normal cells, or the mammal as a whole.

In one example, the pharmaceutically effective amount of the compound of the invention administered parenterally per dose will be in the range of about 0.01 to 1000 mg/kg, alternatively about 0.01 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 1 to 500 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of a hyperproliferative disease. Another embodiment includes a pharmaceutical composition comprising a compound of formula (I) for use in the treatment of cancer.

The following embodiments illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention target the TLR7. Accordingly, the compounds of the invention are useful for promoting the activation of antigen presenting cells (APC), contributing to the priming, trafficking and infiltration of T cells, promoting Th1 and CD8+T response, and enhancing the killing of cancer. Compounds of the invention are useful for boosting innate immune response in myeloid cells that express TLR7. Alternatively, compounds of the invention are useful for invigorate T cells in tumors in which the T cell present but suppressed, for example by upregulation of co-stimulatory molecules and production of pro-inflammatory cytokines. More broadly, the compounds can be used for the treatment of cancer types which are non-inflamed, e.g. immune desert or immune excluded by enhancing antigen presentation, T cell priming, recruitment/infiltration, and tumor killing.

Another embodiment includes a method of treating or preventing cancer in a mammal in need of such treatment, wherein the method comprises administering to said mammal a therapeutically effective amount of a compound of formula (I), a stereoisomer, tautomer, or pharmaceutically acceptable salt thereof. Cancer herein includes but is not limited to advanced solid tumors such as pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma, squamous cell carcinoma.

### Synthesis

The compounds of the present invention can be prepared by any conventional means. Suitable processes for synthesizing these compounds as well as their starting materials are provided in the schemes below and in the examples. All substituents, in particular, R¹ to R⁴ are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

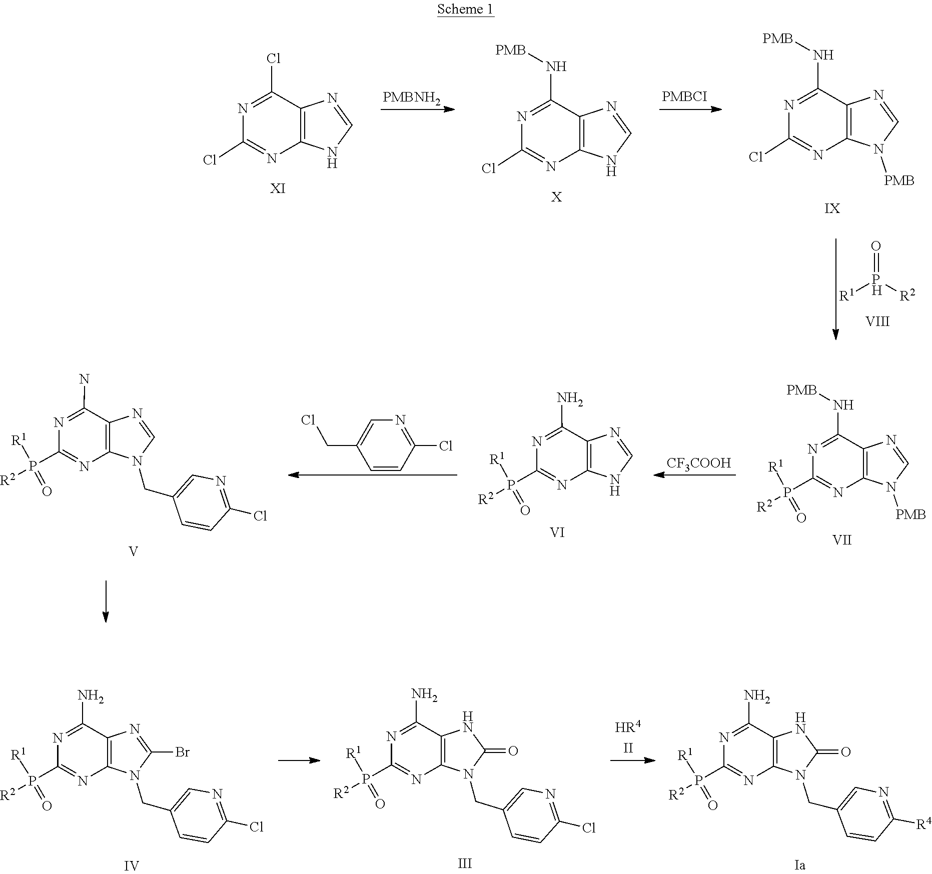

A compound of formula (X) is prepared by reaction of a compound of formula (XI) with (4-methoxyphenyl)methanamine in the presence of organic base, such as trimethylamine or N,N-diisopropylethylamine. A compound of formula (IX) is prepared by reaction of a compound of formula (X) with 1-(chloromethyl)-4-methoxy-benzene in the presence of inorganic base, such as potassium carbonate, sodium hydride or cesium carbonate. A compound of formula (VII) is prepared by reaction of a compound of formula (IX) with a compound of formula (VIII) in the presence of organometallic catalyst system, such as Pd(OAc)₂/XantPhos/K₂CO₃ or Pd₂(dba)₃/XantPhos/TEA. A compound of formula (V) is prepared by deprotection of a compound of formula (VII) with acid, such as trifluoroacetic acid, followed by alkylation with 2-chloro-5-(chloromethyl)pyridine in the presence of inorganic base, such as potassium carbonate, sodium hydride or cesium carbonate. A compound of formula (III) is prepared by bromination of a compound of formula (V) with brominating agent, such as bromine or N-bromosuccinimide, followed by hydrolysis with sodium hydroxide aqueous solution. A compound of formula (Ia) is prepared by reaction of a compound of formula (III) with an amine or alcohol HR⁴, compound of formula (II), in a neat reaction, or in the presence of inorganic base, such as sodium hydride, or in the presence of organometallic catalyst system, such as Pd₂(dba)₃/RuPhos/t-BuONa, Pd₂(dba)₃/BrettPhos/t-BuONa, or Pd-PEPPSI-IPentCl/t-BuOK.

Compounds of this invention can be obtained as mixtures of diastereomers or enantiomers, which can be separated by methods well known in the art, e.g. (chiral) HPLC or SFC. In another embodiment, compound of formula (I) or (Ia) can be obtained according to above scheme by using corresponding chiral starting materials.

This invention also relates to a process for the preparation of a compound of formula (I) or (Ia) comprising the following step:
a) reaction between compound of formula (III),

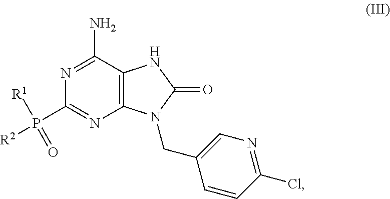

and an amine or alcohol HR⁴ (II), in a neat reaction, or in the presence of inorganic base, such as sodium hydride, or in the presence of organometallic catalyst system, such as Pd₂(dba)₃/RuPhos/t-BuONa, Pd₂(dba)₃/BrettPhos/t-BuONa, or Pd-PEPPSI-IPentCl/t-BuOK.

A compound of formula (I) when manufactured according to the above process is also an object of the invention.

### EXAMPLES

The invention will be more fully understood by reference to the following examples. They should not, however, be construed as limiting the scope of the invention.

### Abbreviations

aq. Aqueous
BrettPhos: dicyclohexyl-[3,6-dimethoxy-2-(2,4,6-triisopropylphenyl)phenyl]phosphane
BSA: N, O-bis(trimethylsilyl)acetamide
CDI: N,N′-carbonyl diimidazole
DIEPA: N,N-diethylpropylamine
DMF: dimethyl formamide
DBU: 1,8-diazabicycloundec-7-ene
DPPA: diphenylphosphoryl azide
EC₅₀: the molar concentration of an agonist, which produces 50% of the maximum possible response for that agonist.
EDC: N1-((ethylimino)methylene)-N3,N3-dimethylpropane-1,3-diamine
eq: equivalent
EtOAc or EA: ethyl acetate
HATU: (1-[bis(dimethylamino)methylene]-1H-1,2,3-triazolo[4,5-b]pyridinium 3-oxid hexafluorophosphate)
hr(s): hour(s)
HPLC: high performance liquid chromatography
HOBt: N-hydroxybenzotriazole
MS (ESI): mass spectroscopy (electron spray ionization)
m-CPBA: 3-chloroperbenzoic acid
MTEB: methyl tert-butyl ether
NMR: nuclear magnetic resonance
NMP: N-methylpyrrolidone
obsd. Observed
Pd-PEPPSI-IPentCl: (SP-4-1)-[1,3-bis[2,6-bis(1-ethylpropyl)phenyl]-4,5-dichloro-1,3-dihydro-2H-imidazol-2-ylidene]dichloro(2-methylpyridine)palladium
PE: petroleum ether
PMB: p-methoxybenzyl
PPA: polyphosphoric acid
RT or rt: room temperature
RuPhos: dicyclohexyl-[2-(2,6-diisopropoxyphenyl)phenyl]phosphane
sat. saturated
SFC: supercritical fluid chromatography
TEA: triethylamine
TFA: trifluoroacetic acid
THF: tetrahydrofuran
TLC: thin layer chromatography
V/V: volume ratio
XantPhos: (9,9-dimethyl-9H-xanthene-4,5-diyl)bis(diphenylphosphine)

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using Phenomenex Luna C18 (15 μm, 150×400 mm) column, Phenomenex Luna C18 (10 μm, 100×250 mm) column, Phenomenex Luna C18 (10 μm, 80×250 mm) column, Shim-pack C18 (10 μm, 25×150 mm) column, Phenomenex Synergi C18 (10 μm, 25×150 mm) column, or Waters Atlantis T3 (5 μm, 30×150 mm) column. Waters AutoP purification System (Sample Manager 2767, Pump 2525, Detector: Micromass ZQ and UV 2487, solvent system: acetonitrile and 0.1% ammonium bicarbonate in water; acetonitrile and 0.1% FA in water or acetonitrile and 0.1% TFA in water). Or Gilson-281 purification System (Pump 322, Detector: UV 156, solvent system: acetonitrile and 0.05% ammonium bicarbonate in water; acetonitrile and 0.225% FA in water; acetonitrile and 0.05% HCl in water; acetonitrile and 0.075% TFA in water; or acetonitrile and water).

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz or Bruker Avance 500 MHz.

All reactions involving air-sensitive reagents were performed under an argon atmosphere. Reagents were used as received from commercial suppliers without further purification unless otherwise noted.

### PREPARATIVE EXAMPLES

### Example 1

### 6-amino-9-[[6-[2-(dimethylamino)ethylamino]-3-pyridyl]methyl]-2-dimethylphosphoryl-7H-purin-8-one

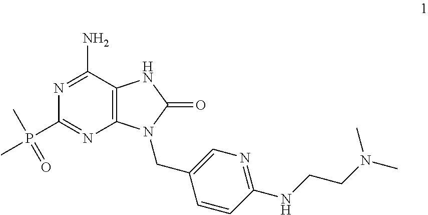

### Step 1: Preparation of 2-chloro-N-[(4-methoxyphenyl)methyl]-9H-purin-6-amine (Compound 1a)

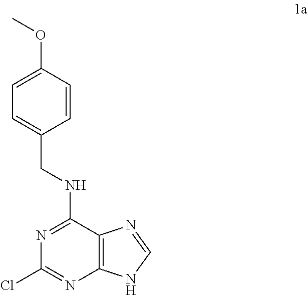 [SMILES: COc1ccc(CNc2nc(Cl)nc3[nH]cnc23)cc1]

To a solution of 2,6-dichloro-9H-purine (50.0 g, 264.55 mmol, 1 eq) and TEA (73.63 mL, 529.7 mmol, 2 eq) in DMF (400 mL) was added (4-methoxyphenyl)methanamine (36.29 mL, 277.78 mmol, 1.05 eq). The mixture was stirred at 100° C. for 16 hrs. The mixture was cooled to 15° C., then filtered. The filtered cake was washed with EA (3×150 mL) and H₂O (3×150 mL). The residue was dried under reduced pressure to give 2-chloro-N-[(4-methoxyphenyl)methyl]-9H-purin-6-amine (60 g, Compound 1a) as a yellow solid. ¹H NMR (400 MHz, DMSO-d₆) δ ppm 8.55 (br s, 1H), 8.13 (s, 1H), 7.29 (d, J=8.8 Hz, 2H), 6.89 (d, J=8.4 Hz, 2H), 4.56 (s, 2H), 3.71 (s, 3H).

### Step 2: Preparation of 2-chloro-N,9-bis[(4-methoxyphenyl)methyl]purin-6-amine (Compound 1b)

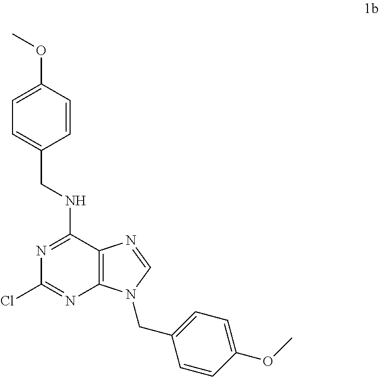 [SMILES: COc1ccc(CNc2nc(Cl)nc3c2ncn3Cc2ccc(OC)cc2)cc1]

To a solution of 2-chloro-N-[(4-methoxyphenyl)methyl]-9H-purin-6-amine (180.0 g, Compound 1a, 621.29 mmol, 1 eq) in DMF (1.8 L) was added K₂CO₃ (89.0 g, 683.42 mmol, 1.1 eq) and 1-(chloromethyl)-4-methoxy-benzene (84.24 mL, 621.29 mmol, 1 eq). The mixture was stirred at 15° C. for 16 hrs and then diluted with H₂O (2.0 L). After filtration, the filter cake was purified by trituration (PE/EA=5/1, 2×600 mL) and dried in vacuo to afford 2-chloro-N,9-bis[(4-methoxyphenyl)methyl]purin-6-amine (180 g, Compound 1b) as a pink solid. MS obsd. (ESI⁺) [{³⁵Cl}(M+H)⁺]: 410.1. ¹H NMR (400 MHz, DMSO-d₆) δ ppm 8.78-8.75 (m, 1H), 8.23 (s, 1H), 7.27 (d, J=8.4 Hz, 4H), 6.91-6.85 (m, 4H), 5.25 (s, 2H), 5.06-4.54 (m, 2H), 3.74-3.69 (m, 6H).

### Step 3: Preparation of 2-dimethylphosphoryl-N,9-bis[(4-methoxyphenyl)methyl]purin-6-amine (Compound 1c)

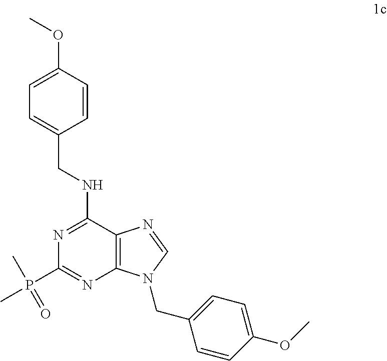

To a solution of 2-chloro-N,9-bis[(4-methoxyphenyl)methyl]purin-6-amine (35 g, Compound 1b, 85.4 mmol, 1 eq) and methylphosphonoylmethane (10.0 g, 128.09 mmol, 1.5 eq) in DMF (500 mL) was added K₂CO₃ (23.6 g, 170.79 mmol, 2 eq), XantPhos (4.9 g, 8.54 mmol, 0.1 eq) and Pd(OAc)₂ (1.9 g, 8.54 mmol, 0.1 eq). The mixture was stirred at 150° C. for 2 hrs under N2 atmosphere. The mixture was filtered through celite and the filtrate was concentrated in vacuo to afford 2-dimethylphosphoryl-N,9-bis[(4-methoxyphenyl)methyl]purin-6-amine (76 g, Compound 1c, crude) as a yellow oil which was used in next step without further purification. MS obsd. (ESI⁺) [(M+H)⁺]: 452.2.

### Step 4: Preparation of 2-dimethylphosphoryl-9H-purin-6-amine (Compound 1d)

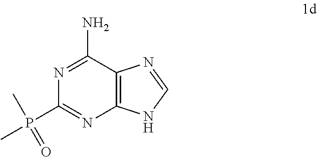

A solution of 2-dimethylphosphoryl-N,9-bis[(4-methoxyphenyl)methyl]purin-6-amine (76.0 g, Compound 1c, crude) in TFA (760.0 mL) was stirred at 60° C. for 16 hrs. The mixture was concentrated in vacuo and the residue was triturated with MeOH (3×400 mL) and EA (3×60 mL) to afford 2-dimethylphosphoryl-9H-purin-6-amine (30 g, Compound 1d, crude) as a light yellow solid and used in next step without further purification. MS obsd. (ESI⁺) [(M+H)⁺]: 211.0.

### Step 5: Preparation of 9-[(6-chloro-3-pyridyl)methyl]-2-dimethylphosphoryl-purin-6-amine (Compound 1e)

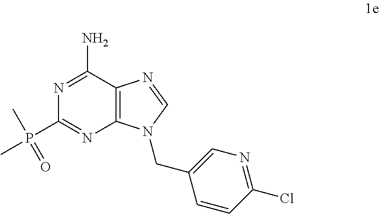 [SMILES: CP(C)(=O)c1nc(N)c2ncn(Cc3ccc(Cl)nc3)c2n1]

To a solution of 2-dimethylphosphoryl-9H-purin-6-amine (30.0 g, Compound 1d, 142.07 mmol, 1 eq) in DMF (300 mL) was added K₂CO₃ (37.0 g, 284.14 mmol, 2 eq) and 2-chloro-5-(chloromethyl)pyridine (23.0 g, 142.07 mmol, 1 eq). The mixture stirred at 40° C. for 16 hrs. The mixture was filtered. The filtrate was concentrated in vacuo and the residue was purified by prep-HPLC to afford 9-[(6-chloro-3-pyridyl)methyl]-2-dimethylphosphoryl-purin-6-amine (22 g, Compound 1e) as a light yellow solid. MS obsd. (ESI⁺) [{³⁵Cl}(M+H)⁺]: 337.0. ¹H NMR (400 MHz, DMSO-d₆) δ ppm 8.55 (d, J=2.0 Hz, 1H), 8.45 (s, 1H), 7.90 (dd, J=8.8, 2.8 Hz, 1H), 7.72 (br s, 2H), 7.52 (d, J=8.4 Hz, 1H), 5.46 (s, 2H), 1.72 (d, J=13.6 Hz, 6H).

### Step 6: Preparation of 8-bromo-9-[(6-chloro-3-pyridyl)methyl]-2-dimethylphosphoryl-purin-6-amine (Compound 1f)

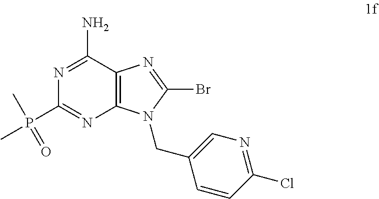 [SMILES: CP(C)(=O)c1nc(N)c2nc(Br)n(Cc3ccc(Cl)nc3)c2n1]

To a solution of 9-[(6-chloro-3-pyridyl)methyl]-2-dimethylphosphoryl-purin-6-amine (22.0 g, Compound 1e, 65.34 mmol, 1 eq), NaOAc (11.8 g, 143.74 mmol, 2.2 eq) and AcOH (3.9 g, 65.34 mmol, 1 eq) in water (220 mL) was added Br₂ (20.9 g, 130.67 mmol, 2 eq) dropwise at 15° C. Then the mixture was stirred at 15° C. for 16 hrs. The mixture was extracted with DCM/MeOH (v/v=10/1, 3×200 mL). The organic layers were combined and dried over Na₂SO₄. After filtration, the filtrate was concentrated in vacuo and the residue was purified by prep-HPLC to afford 8-bromo-9-[(6-chloro-3-pyridyl)methyl]-2-dimethylphosphoryl-purin-6-amine (11 g, Compound 1f) as a red solid. MS obsd. (ESI⁺) [{³⁵Cl&⁷⁹Br}(M+H)⁺]: 416.9. ¹H NMR (400 MHz, DMSO-d₆) δ ppm 8.48 (s, 1H), 7.87-7.68 (m, 3H), 7.53 (d, J=8.0 Hz, 1H), 5.44 (s 2H), 1.71 (d, J=13.6 Hz, 6H).

### Step 7: Preparation of 6-amino-9-[(6-chloro-3-pyridyl)methyl]-2-dimethylphosphoryl-7H-purin-8-one (Compound 1g)

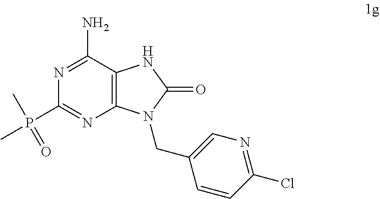 [SMILES: CP(C)(=O)c1nc(N)c2[nH]c(=O)n(Cc3ccc(Cl)nc3)c2n1]

A solution of 8-bromo-9-[(6-chloro-3-pyridyl)methyl]-2-dimethylphosphoryl-purin-6-amine (11.5 g, Compound 1f, 27.67 mmol, 1 eq) and NaOH (8.8 g, 221.36 mmol, 8 eq) in water (80 mL) and tert-butanol (80 mL) was stirred at 80° C. for 16 hrs. The mixture was concentrated in vacuo, then diluted with H₂O (80 mL). HCl aq. (6M) was added to bring pH=8. After filtration, the filter cake was washed with H₂O (3×60 mL) and dried in vacuo. The residue was purified by trituration (EA/MeOH=10/1, 3×20 mL) to afford 6-amino-9-[(6-chloro-3-pyridyl)methyl]-2-dimethylphosphoryl-7H-purin-8-one (8.5 g, Compound 1g) as a grey solid which was used in next step without further purification. MS obsd. (ESI⁺) [{³⁵Cl}(M+H)⁺]: 353.0. ¹H NMR (400 MHz, DMSO-d₆) δ ppm 8.45 (d, J=2.0 Hz, 1H), 7.82 (dd, J=8.0, 2.4 Hz 1H), 7.50 (d, J=8.0 Hz, 1H), 6.74 (br s, 2H), 4.99 (s, 2H), 1.67 (d, J=13.6 Hz, 6H).

### Step 8: Preparation of 6-amino-9-[[6-[2-(dimethylamino)ethylamino]-3-pyridyl]methyl]-2-dimethylphosphoryl-7H-purin-8-one (Example 1)

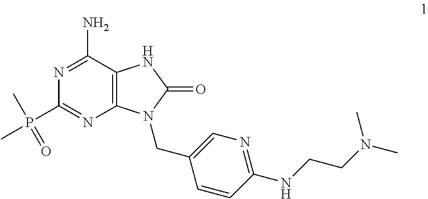

A mixture of 6-amino-9-[(6-chloro-3-pyridyl)methyl]-2-dimethylphosphoryl-7H-purin-8-one (7.5 g, Compound 1g, 21.26 mmol, 1 eq) in N′,N′-dimethylethane-1,2-diamine (90.0 mL, 212.63 mmol, 10 eq) was stirred at 170° C. for 72 hrs in a sealed tube. The reaction mixture was concentrated in vacuo and the residue was purified by prep-HPLC. The crude product was dissolved in H₂O/MeOH=1/1 (60 mL) and adjusted to pH=8 by adding NaHCO₃ solid and then concentrated in vacuo. The residue was dissolved in DCM/MeOH=5/1 (100 mL) and the mixture was filtered and then, the filtrate was concentrated in vacuo. The residue was dissolved in MeCN/MeOH=20/1 (70 mL) and stirred at 85° C. for 2 hrs and then cooled to 15° C. After filtration, the filter cake was dried in vacuo to afford 6-amino-9-[[6-[2-(dimethylamino)ethylamino]-3-pyridyl]methyl]-2-dimethylphosphoryl-7H-purin-8-one (2.2 g, Example 1) as a white solid. MS obsd. (ESI⁺) [(M+H)⁺]: 405.1. ¹H NMR (500 MHz, DMSO-d₆) δ ppm 10.49 (br d, J=2.0 Hz, 1H), 8.01 (d, J=2.1 Hz, 1H), 7.39 (dd, J=8.6, 2.4 Hz, 1H), 6.70 (br s, 2H), 6.42 (d, J=8.7 Hz, 1H), 6.36 (t, J=5.4 Hz, 1H), 4.75 (s, 2H), 3.27 (q, J=6.5 Hz, 2H), 2.35 (t, J=6.6 Hz, 2H), 2.14 (s, 6H), 1.68 (d, J=13.6 Hz, 6H).

### Example 2

### 6-amino-9-[[6-[2-(dimethylamino)ethoxy]-3-pyridyl]methyl]-2-dimethylphosphoryl-7H-purin-8-one

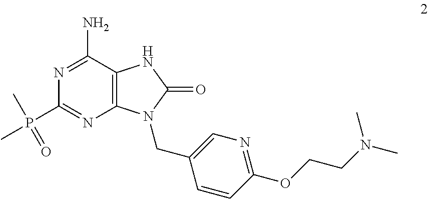

To a solution of 2-(dimethylamino)ethanol (126.36 mg, 1.42 mmol, 10 eq) in THF (3 mL) was added NaH (56.7 mg, 60 wt. %, 1.42 mmol, 10 eq) at 0° C. The mixture was stirred at 0° C. for 1 h. Then the mixture was concentrated in vacuo. The residue was dissolved in DMF (2 mL) and 6-amino-9-[(6-chloro-3-pyridyl)methyl]-2-dimethylphosphoryl-7H-purin-8-one (50.0 mg, Compound 1g, 0.140 mmol, 1 eq) was added. The mixture was stirred at 130° C. for 15 hrs. H₂O (10 mL) was added dropwise at 0° C. and then FA was added to bring pH=6. Then the mixture was concentrated in vacuo and the residue was purified by prep-HPLC to afford 6-amino-9-[[6-[2-(dimethylamino)ethoxy]-3-pyridyl]methyl]-2-dimethylphosphoryl-7H-purin-8-one (12.6 mg, Example 2) as a light yellow solid. MS obsd. (ESI⁺) [(M+H)⁺]. 406.1. ¹H NMR (400 MHz, CD₃OD) δ ppm 8.26 (d, J=2.4 Hz, 1H), 7.81 (dd, J=8.8, 2.8 Hz, 1H), 6.77 (d, J=8.4 Hz, 1H), 5.02 (s, 2H), 4.38 (t, J=5.6 Hz, 2H), 2.75 (t, J=5.6 Hz, 2H), 2.31 (s, 6H), 1.85 (d, J=13.6 Hz, 6H).

### Example 3

### 6-amino-9-[[6-(3-aminopyrrolidin-1-yl)-3-pyridyl]methyl]-2-dimethylphosphoryl-7H-purin-8-one

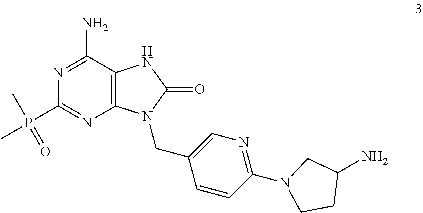 [SMILES: CP(C)(=O)c1nc(N)c2[nH]c(=O)n(Cc3ccc(N4CCC(N)C4)nc3)c2n1]

### Step 1: Preparation of tert-butyl N-[1-[5-[(6-amino-2-dimethylphosphoryl-8-oxo-7H-purin-9-yl)methyl]-2-pyridyl]pyrrolidin-3-yl]carbamate (Compound 3a)

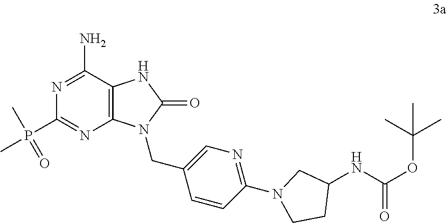 [SMILES: CC(C)(C)OC(=O)NC1CCN(c2ccc(Cn3c(=O)[nH]c4c(N)nc(P(C)(C)=O)nc43)cn2)C1]

To a mixture of 6-amino-9-[(6-chloro-3-pyridyl)methyl]-2-dimethylphosphoryl-7H-purin-8-one (100.0 mg, Compound 1g, 0.280 mmol, 1 eq), tert-butyl N-pyrrolidin-3-ylcarbamate (79.2 mg, 0.430 mmol, 1.5 eq) and t-BuONa (54.4 mg, 0.570 mmol, 2 eq) in NMP (2 mL) were added RuPhos (13.2 mg, 0.030 mmol, 0.1 eq) and Pd₂(dba)₃ (25.9 mg, 0.030 mmol, 0.1 eq). The mixture was stirred at 120° C. for 16 h under N₂ atmosphere. The mixture was filtered. The filtrate was concentrated in vacuo and purified by prep-HPLC to afford tert-butyl N-[1-[5-[(6-amino-2-dimethylphosphoryl-8-oxo-7H-purin-9-yl)methyl]-2-pyridyl]pyrrolidin-3-yl]carbamate (15 mg, Compound 3a) as a light yellow solid. MS obsd. (ESI⁺) [(M+H)⁺]: 503.2.

### Step 2: Preparation of 6-amino-9-[[6-(3-aminopyrrolidin-1-yl)-3-pyridyl]methyl]-2-dimethylphosphoryl-7H-purin-8-one (Example 3)

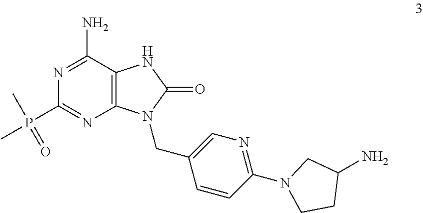 [SMILES: CP(C)(=O)c1nc(N)c2[nH]c(=O)n(Cc3ccc(N4CCC(N)C4)nc3)c2n1]

A solution of tert-butyl N-[1-[5-[(6-amino-2-dimethylphosphoryl-8-oxo-7H-purin-9-yl)methyl]-2-pyridyl]pyrrolidin-3-yl]carbamate (15.0 mg, Compound 3a, 0.030 mmol, 1 eq) in HCl/dioxane (1.0 mL, 4 M) was stirred at 15° C. for 1 h. The reaction mixture was concentrated in vacuo. The residue was purified by prep-HPLC to afford 6-amino-9-[[6-(3-aminopyrrolidin-1-yl)-3-pyridyl]methyl]-2-dimethylphosphoryl-7H-purin-8-one (8.7 mg, Example 3) as a light yellow solid. MS obsd. (ESI⁺) [(M+H)⁺]: 402.9. ¹H NMR (400 MHz, CD₃OD) δ ppm 8.17 (d, J=2.0 Hz, 1H), 7.69-7.65 (m, 1H), 6.46 (d, J=8.4 Hz, 1H), 4.99 (s, 2H), 3.68-3.63 (m, 3H), 3.57-3.48 (m, 1H), 3.31-3.30 (m, 1H), 2.27-2.22 (m, 1H), 1.91-1.89 (m, 1H), 1.84 (d, J=13.6 Hz, 6H).

### Example 4

### 6-amino-2-dimethylphosphoryl-9-[[6-(3,3-dimethylpiperazin-1-yl)-3-pyridyl]methyl]-7H-purin-8-one

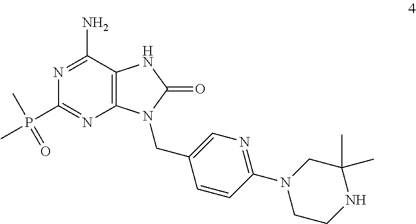

### Step 1: Preparation of tert-butyl 4-[5-[(6-amino-2-dimethylphosphoryl-8-oxo-7H-purin-9-yl)methyl]-2-pyridyl]-2,2-dimethyl-piperazine-1-carboxylate (Compound 4a)

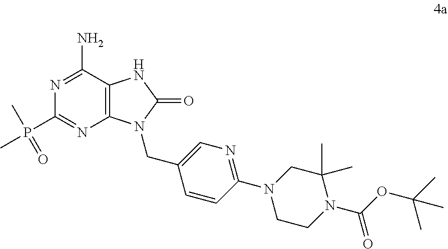 [SMILES: CC(C)(C)OC(=O)N1CCN(c2ccc(Cn3c(=O)[nH]c4c(N)nc(P(C)(C)=O)nc43)cn2)CC1(C)C]

The title compound was prepared in analogy to Example 3, Step 1 by using tert-butyl 2,2-dimethylpiperazine-1-carboxylate instead of tert-butyl N-pyrrolidin-3-ylcarbamate. tert-Butyl 4-[5-[(6-amino-2-dimethylphosphoryl-8-oxo-7H-purin-9-yl)methyl]-2-pyridyl]-2,2-dimethyl-piperazine-1-carboxylate (80 mg, Compound 4a) was obtained as a light yellow oil. MS obsd. (ESI⁺) [(M+H)⁺]: 531.0.

### Step 2: Preparation of 6-amino-2-dimethylphosphoryl-9-[[6-(3,3-dimethylpiperazin-1-yl)-3-pyridyl]methyl]-7H-purin-8-one (Example 4)

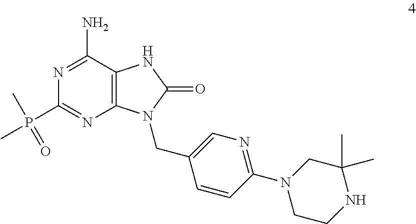

The title compound was prepared in analogy to Example 3, Step 2 by using tert-butyl 4-[5-[(6-amino-2-dimethylphosphoryl-8-oxo-7H-purin-9-yl)methyl]-2-pyridyl]-2,2-dimethyl-piperazine-1-carboxylate (Compound 4a) instead of tert-butyl N-[1-[5-[(6-amino-2-dimethylphosphoryl-8-oxo-7H-purin-9-yl)methyl]-2-pyridyl]pyrrolidin-3-yl]carbamate (Compound 3a). 6-Amino-2-dimethylphosphoryl-9-[[6-(3,3-dimethylpiperazin-1-yl)-3-pyridyl]methyl]-7H-purin-8-one (6.1 mg, Example 4) was obtained as a white solid. MS obsd. (ESI⁺) [(M+H)⁺]: 431.2. ¹H NMR (400 MHz, CD₃OD) δ ppm 8.30 (d, J=2.0 Hz, 1H), 7.71 (dd, J=8.8, 2.4 Hz, 1H), 6.88 (d, J=8.8 Hz, 1H), 5.00 (s, 2H), 3.77 (t, J=5.6 Hz, 2H), 3.60 (s, 2H), 3.32-3.30 (m, 2H), 1.88 (d, J=13.6 Hz, 6H), 1.40 (s, 6H).

### Example 5

### 6-amino-9-[[6-(2,5-diazabicyclo[2.2.2]octan-2-yl)-3-pyridyl]methyl]-2-dimethylphosphoryl-7H-purin-8-one

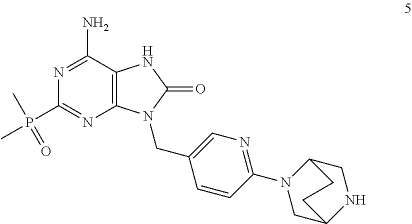 [SMILES: CP(C)(=O)c1nc(N)c2[nH]c(=O)n(Cc3ccc(N4CC5CCC4CN5)nc3)c2n1]

### Step 1: Preparation of tert-butyl 5-[5-[(6-amino-2-dimethylphosphoryl-8-oxo-7H-purin-9-yl)methyl]-2-pyridyl]-2,5-diazabicyclo[2.2.2]octane-2-carboxylate (Compound 5a)

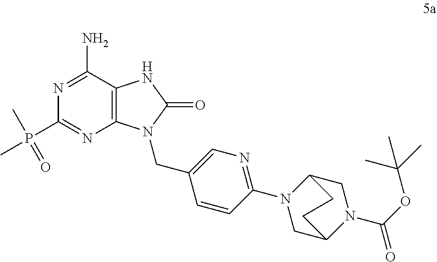 [SMILES: CC(C)(C)OC(=O)N1CC2CCC1CN2c1ccc(Cn2c(=O)[nH]c3c(N)nc(P(C)(C)=O)nc32)cn1]

The title compound was prepared in analogy to Example 3, Step 1 by using tert-butyl 2,5-diazabicyclo[2.2.2]octane-2-carboxylate instead of tert-butyl N-pyrrolidin-3-ylcarbamate. tert-Butyl 5-[5-[(6-amino-2-dimethylphosphoryl-8-oxo-7H-purin-9-yl)methyl]-2-pyridyl]-2,5-diazabicyclo[2.2.2]octane-2-carboxylate (35 mg, Compound 5a) was obtained as a brown solid. MS obsd. (ESI⁺) [(M+H)⁺]: 529.1.

### Step 2: Preparation of 6-amino-9-[[6-(2,5-diazabicyclo[2.2.2]octan-2-yl)-3-pyridyl]methyl]-2-dimethylphosphoryl-7H-purin-8-one (Example 5)

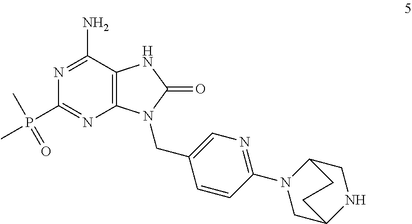 [SMILES: CP(C)(=O)c1nc(N)c2[nH]c(=O)n(Cc3ccc(N4CC5CCC4CN5)nc3)c2n1]

The title compound was prepared in analogy to Example 3, Step 2 by using tert-butyl 5-[5-[(6-amino-2-dimethylphosphoryl-8-oxo-7H-purin-9-yl)methyl]-2-pyridyl]-2,5-diazabicyclo[2.2.2]octane-2-carboxylate (Compound 5a) instead of tert-butyl N-[1-[5-[(6-amino-2-dimethylphosphoryl-8-oxo-7H-purin-9-yl)methyl]-2-pyridyl]pyrrolidin-3-yl]carbamate (Compound 3a). 6-Amino-9-[[6-(2,5-diazabicyclo[2.2.2]octan-2-yl)-3-pyridyl]methyl]-2-dimethylphosphoryl-7H-purin-8-one (9.3 mg, Example 5) was obtained as a green solid. MS obsd. (ESI⁺) [(M+H)⁺]: 428.9. ¹H NMR (400 MHz, CD₃OD) δ ppm 8.26 (s, 1H), 7.75 (d, J=7.6 Hz, 1H), 6.60 (d, J=8.8 Hz, 1H), 4.98 (s, 2H), 3.82-3.77 (m, 2H), 3.44-3.43 (m, 4H), 2.14-2.04 (m, 4H), 1.88 (d, J=13.6 Hz, 6H).

### Example 6

### 9-[[6-(2,3,3a,4,6,6a-hexahydro-1H-pyrrolo[3,4-c]pyrrol-5-yl)-3-pyridyl]methyl]-6-amino-2-dimethylphosphoryl-7H-purin-8-one

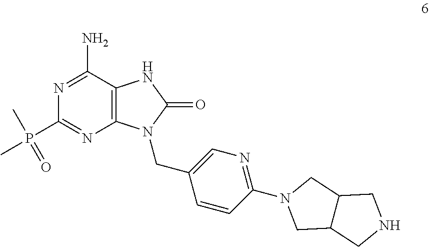 [SMILES: CP(C)(=O)c1nc(N)c2[nH]c(=O)n(Cc3ccc(N4CC5CNCC5C4)nc3)c2n1]

### Step 1: Preparation of tert-butyl 2-[5-[(6-amino-2-dimethylphosphoryl-8-oxo-7H-purin-9-yl)methyl]-2-pyridyl]-1,3,3a,4,6,6a-hexahydropyrrolo[3,4-c]pyrrole-5-carboxylate (Compound 6a)

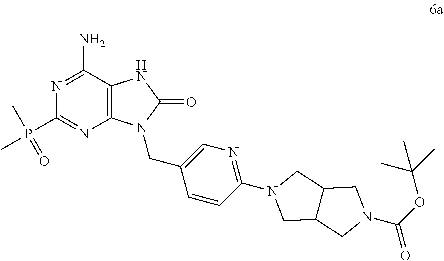 [SMILES: CC(C)(C)OC(=O)N1CC2CN(c3ccc(Cn4c(=O)[nH]c5c(N)nc(P(C)(C)=O)nc54)cn3)CC2C1]

The title compound was prepared in analogy to Example 3, Step 1 by using tert-butyl 2,3,3a,4,6,6a-hexahydro-1H-pyrrolo[3,4-c]pyrrole-5-carboxylate instead of tert-butyl N-pyrrolidin-3-ylcarbamate. tert-Butyl 2-[5-[(6-amino-2-dimethylphosphoryl-8-oxo-7H-purin-9-yl)methyl]-2-pyridyl]-1,3,3a,4,6,6a-hexahydropyrrolo[3,4-c]pyrrole-5-carboxylate (80 mg, Compound 6a) was obtained as a light yellow oil. MS obsd. (ESI⁺) [(M+H)⁺]: 529.0.

### Step 2: Preparation of 9-[[6-(2,3,3a,4,6,6a-hexahydro-1H-pyrrolo[3,4-c]pyrrol-5-yl)-3-pyridyl]methyl]-6-amino-2-dimethylphosphoryl-7H-purin-8-one (Example 6)

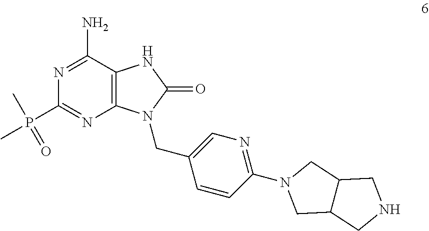 [SMILES: CP(C)(=O)c1nc(N)c2[nH]c(=O)n(Cc3ccc(N4CC5CNCC5C4)nc3)c2n1]

The title compound was prepared in analogy to Example 3, Step 2 by using tert-butyl 2-[5-[(6-amino-2-dimethylphosphoryl-8-oxo-7H-purin-9-yl)methyl]-2-pyridyl]-1,3,3a,4,6,6a-hexahydropyrrolo[3,4-c]pyrrole-5-carboxylate (Compound 6a) instead of tert-butyl N-[1-[5-[(6-amino-2-dimethylphosphoryl-8-oxo-7H-purin-9-yl)methyl]-2-pyridyl]pyrrolidin-3-yl]carbamate (Compound 3a). 9-[[6-(2,3,3a,4,6,6a-Hexahydro-1H-pyrrolo[3,4-c]pyrrol-5-yl)-3-pyridyl]methyl]-6-amino-2-dimethylphosphoryl-7H-purin-8-one (28 mg, Example 6) was obtained as a white solid. MS obsd. (ESI⁺) [(M+H)⁺]: 429.0. ¹H NMR (400 MHz, CD₃OD) δ ppm 8.25 (d, J=2.0 Hz, 1H), 7.75 (dd, J=8.8, 2.4 Hz, 1H), 6.60 (d, J=8.8 Hz, 1H), 5.04 (s, 2H), 3.59-3.55 (m, 6H), 3.33-3.29 (m, 4H), 1.84 (d, J=13.6 Hz, 6H).

### Example 7

### 6-amino-2-dimethylphosphoryl-9-[[6-(2-pyrrolidin-1-ylethylamino)-3-pyridyl]methyl]-7H-purin-8-one

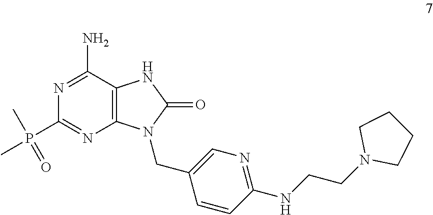 [SMILES: CP(C)(=O)c1nc(N)c2[nH]c(=O)n(Cc3ccc(NCCN4CCCC4)nc3)c2n1]

The title compound was prepared in analogy to Example 1, Step 8 by using 2-pyrrolidin-1-ylethanamine instead of N′,N′-dimethylethane-1,2-diamine. 6-Amino-2-dimethylphosphoryl-9-[[6-(2-pyrrolidin-1-ylethylamino)-3-pyridyl]methyl]-7H-purin-8-one (13.3 mg, Example 7) was obtained as a white solid. MS obsd. (ESI⁺) [(M+H)⁺]: 431.1. ¹H NMR (400 MHz, CD₃OD) δ ppm 8.08 (d, J=2.0 Hz, 1H), 7.57 (dd, J=8.8, 2.4 Hz, 1H), 6.46 (d, J=8.4 Hz, 1H), 4.92 (s, 2H), 3.41 (t, J=6.8 Hz, 2H), 2.69 (t, J=6.8 Hz, 2H), 2.62-2.58 (m, 4H), 1.84 (d, J=13.6 Hz, 6H), 1.83-1.77 (m, 4H).

### Example 8

### 6-amino-2-dimethylphosphoryl-9-[[6-(tetrahydropyran-4-ylamino)-3-pyridyl]methyl]-7H-purin-8-one

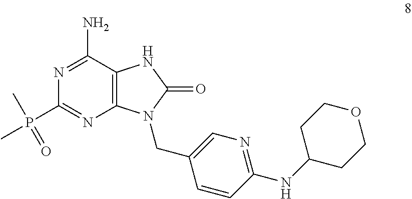 [SMILES: CP(C)(=O)c1nc(N)c2[nH]c(=O)n(Cc3ccc(NC4CCOCC4)nc3)c2n1]

The title compound was prepared in analogy to Example 1, Step 8 by using tetrahydropyran-4-amine instead of N′,N′-dimethylethane-1,2-diamine. 6-Amino-2-dimethylphosphoryl-9-[[6-(tetrahydropyran-4-ylamino)-3-pyridyl]methyl]-7H-purin-8-one (18.4 mg, Example 8) was obtained as a white solid. MS obsd. (ESI⁺) [(M+H)⁺]: 418.1. ¹H NMR (400 MHz, CD₃OD) δ ppm 8.06 (d, J=2.0 Hz, 1H), 7.55 (dd, J=8.8, 2.4 Hz, 1H), 6.46 (d, J=8.4 Hz, 1H), 4.91 (s, 2H), 3.96-3.91 (m, 2H), 3.86-3.83 (m, 1H), 3.55-3.49 (m, 2H), 1.95-1.91 (m, 2H), 1.84 (d, J=13.6 Hz, 6H), 1.50-1.46 (m, 2H).

### Example 9

### 6-amino-9-[[6-[3-[(dimethylamino)methyl]pyrrolidin-1-yl]-3-pyridyl]methyl]-2-dimethylphosphoryl-7H-purin-8-one

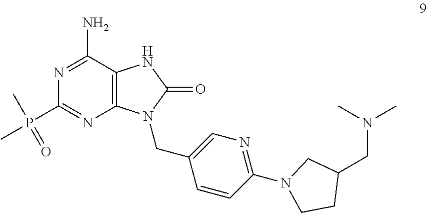

The title compound was prepared in analogy to Example 3, Step 1 by using N,N-dimethyl-1-pyrrolidin-3-yl-methanamine instead of tert-butyl N-pyrrolidin-3-ylcarbamate. 6-Amino-9-[[6-[3-[(dimethylamino)methyl]pyrrolidin-1-yl]-3-pyridyl]methyl]-2-dimethylphosphoryl-7H-purin-8-one (12.1 mg, Example 9) was obtained as a light yellow oil. MS obsd. (ESI⁺) [(M+H)⁺]: 445.0. ¹H NMR (400 MHz, CD₃OD) δ ppm 8.19 (d, J=2.0 Hz, 1H), 7.71 (dd, J=8.4, 2.0 Hz, 1H), 6.51 (d, J=8.8 Hz, 1H), 4.96 (s, 2H), 3.75-3.72 (m, 1H), 3.61-3.55 (m, 1H), 3.46-3.39 (m, 2H), 3.29-3.27 (m, 2H), 3.18-3.13 (m, 1H), 2.94 (s, 6H), 2.83-2.87 (m, 1H), 2.31-2.27 (m, 1H), 1.85 (d, J=13.6 Hz, 6H).

### Example 10

### 6-amino-2-dimethylphosphoryl-9-[[6-(4-methyl-1-oxa-4,9-diazaspiro[5.5]undecan-9-yl)-3-pyridyl]methyl]-7H-purin-8-one

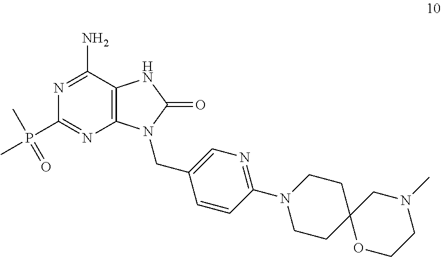

### Step 1: Preparation of 4-methyl-1-oxa-4,9-diazaspiro[5.5]undecane (Compound 10a)

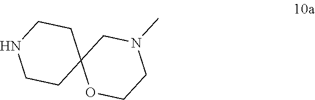

To a solution of tert-butyl 1-oxa-4,9-diazaspiro[5.5]undecane-4-carboxylate (500.0 mg, 1.95 mmol, 1 eq) in THF (10 mL) was added LiAlH₄ (148.05 mg, 3.9 mmol, 2 eq) at 0° C. The reaction mixture was stirred at 80° C. for 2 h. MeOH (2 mL) and H₂O (1 mL) were added at 0° C. The reaction mixture was dried over Na₂SO₄. After filtration, the filtrate was concentrated in vacuo. The residue was purified by prep-HPLC to afford 4-methyl-1-oxa-4,9-diazaspiro[5.5]undecane (200 mg, Compound 10a) as a colorless oil. MS obsd. (ESI⁺) [(M+H)⁺]: 171.1.

### Step 2: Preparation of 6-amino-2-dimethylphosphoryl-9-[[6-(4-methyl-1-oxa-4,9-diazaspiro[5.5]undecan-9-yl)-3-pyridyl]methyl]-7H-purin-8-one (Example 10)

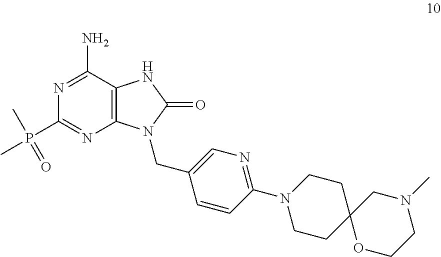

The title compound was prepared in analogy to Example 3, Step 1 by using 4-methyl-1-oxa-4,9-diazaspiro[5.5]undecane instead of tert-butyl N-pyrrolidin-3-ylcarbamate. 6-Amino-2-dimethylphosphoryl-9-[[6-(4-methyl-1-oxa-4,9-diazaspiro[5.5]undecan-9-yl)-3-pyridyl]methyl]-7H-purin-8-one (4.7 mg, Example 10) was obtained as a white solid. MS obsd. (ESI⁺) [(M+H)⁺]: 487.2. ¹H NMR (400 MHz, CD₃OD) δ (ppm) 8.21 (d, J=2.0 Hz, 1H), 7.67 (dd, J=8.8, 2.4 Hz, 1H), 6.78 (d, J=8.8 Hz, 1H), 4.96 (s, 2H), 3.88-3.81 (m, 2H), 3.74 (t, J=4.8 Hz, 2H), 3.28-3.20 (m, 2H), 2.37 (t, J=4.8 Hz, 2H), 2.25 (s, 2H), 2.21 (s, 3H), 1.99-1.92 (m, 2H), 1.84 (d, J=13.6 Hz, 6H), 1.61-1.50 (m, 2H).

### Example 11

### 6-amino-2-dimethylphosphoryl-9-[[6-[(1-methylpyrrolidin-3-yl)amino]-3-pyridyl]methyl]-7H-purin-8-one

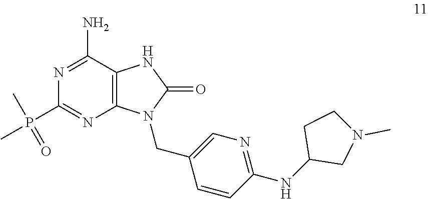

The title compound was prepared in analogy to Example 3, Step 1 by using 1-methylpyrrolidin-3-amine instead of tert-butyl N-pyrrolidin-3-ylcarbamate and BrettPhos instead of RuPhos. 6-amino-2-dimethylphosphoryl-9-[[6-[(1-methylpyrrolidin-3-yl)amino]-3-pyridyl]methyl]-7H-purin-8-one (23.92 mg, Example 11) was obtained as a white solid. MS obsd. (ESI⁺) [(M+H)⁺]: 417.1. ¹H NMR (400 MHz, CD₃OD) δ ppm 8.17 (d, J=2.0 Hz, 1H), 7.62 (dd, J=8.4, 2.0 Hz, 1H), 6.53 (d, J=8.8 Hz, 1H), 4.94 (s, 2H), 4.47-4.44 (m, 1H), 3.68-3.67 (m, 1H), 3.47-3.44 (m, 2H), 3.26-3.23 (m, 1H), 2.93 (s, 3H), 2.52-2.48 (m, 1H), 2.15-2.08 (m, 1H), 1.85 (d, J=13.6 Hz, 6H).

### Example 12

### 6-amino-2-dimethylphosphoryl-9-[[6-[2-[ethyl(methyl)amino]ethylamino]-3-pyridyl]methyl]-7H-purin-8-one

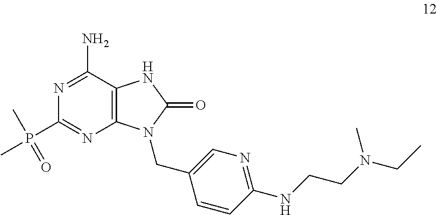 [SMILES: CCN(C)CCNc1ccc(Cn2c(=O)[nH]c3c(N)nc(P(C)(C)=O)nc32)cn1]

### Step 1: Preparation of N′-ethyl-N′-methyl-ethane-1,2-diamine (Compound 12a)

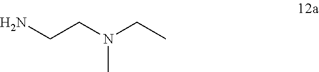

To a solution of N-methylethanamine (14.2 mL, 165.3 mmol, 2.1 eq) in m-Xylene (45 mL) was added 2-(2-bromoethyl)isoindoline-1,3-dione (20.0 g, 78.72 mmol, 1.0 eq) at 20° C. The reaction mixture was stirred at 150° C. for 12 hrs. The reaction mixture was cooled to room temperature, then basified with Na₂CO₃ (aq. 2 M) until pH=9. The mixture was extracted with EtOAc (3×70 mL). The combined organic layer was dried over Na₂SO₄ and filtered. The filtrate was concentrated in vacuo to afford a residue. Then to the residue was added water (5 mL) and HCl (30.0 mL, 12 M, 360.0 mmol, 4.57 eq) at 0° C. The mixture was heated to 130° C. and stirred for 6 hrs when a precipitation formed. The precipitate was filtered off and washed with cold water (50 mL). The filtrate was concentrated in vacuo to afford a residue. The residue was dissolved in MeOH (200 mL), basified to pH=11 with solid NaOH and filtered. The filtrate was concentrated in vacuo and the crude product was purified by distillation at 100° C. (760 mmHg) to afford N′-ethyl-N′-methyl-ethane-1,2-diamine (5.0 g, Compound 12a) as a colorless liquid. ¹H NMR (400 MHz, DMSO-d₆) δ ppm: 2.55 (t, J=6.4 Hz, 2H), 2.30 (q, J=7.2 Hz, 2H), 2.26 (t, J=6.4 Hz, 2H), 2.09 (s, 3H), 0.95 (t, J=7.2 Hz, 3H).

### Step 2: Preparation of 6-amino-2-dimethylphosphoryl-9-[[6-[2-[ethyl(methyl)amino]ethylamino]-3-pyridyl]methyl]-7H-purin-8-one (Example 12)

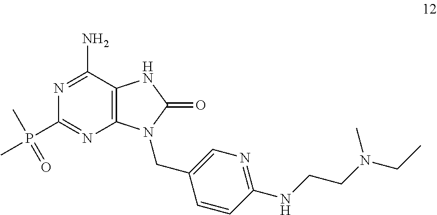 [SMILES: CCN(C)CCNc1ccc(Cn2c(=O)[nH]c3c(N)nc(P(C)(C)=O)nc32)cn1]

The title compound was prepared in analogy to Example 1, Step 8 by using N′-ethyl-N′-methyl-ethane-1,2-diamine instead of N′,N′-dimethylethane-1,2-diamine. 6-Amino-2-dimethylphosphoryl-9-[[6-[2-[ethyl(methyl)amino]ethylamino]-3-pyridyl]methyl]-7H-purin-8-one (8.28 mg, Example 12) was obtained as a white solid. MS obsd. (ESI⁺) [(M+H)⁺]: 419.1. ¹H NMR (400 MHz, CD₃OD) δ ppm: 8.08 (d, J=2.0 Hz, 1H), 7.57 (dd, J 8.8, 2.0 Hz, 1H), 6.46 (d, J=8.4 Hz, 1H), 4.92 (s, 2H), 3.38 (t, J=6.8 Hz, 2H), 2.59 (t, J=6.8 Hz, 2H), 2.50 (q, J=7.2 Hz, 2H), 2.27 (s, 3H), 1.83 (d, J=13.6 Hz, 6H), 1.07 (t, J=7.2 Hz, 3H).

### Example 13-A and Example 13-B

### 6-amino-2-[(R)-ethyl(methyl)phosphoryl]-9-[[6-(4-methylpiperazin-1-yl)-3-pyridyl]methyl]-7H-purin-8-one (Example 13-A) and 6-amino-2-[(S)-ethyl(methyl)phosphoryl]-9-[[6-(4-methylpiperazin-1-yl)-3-pyridyl]methyl]-7H-purin-8-one (Example 13-B)

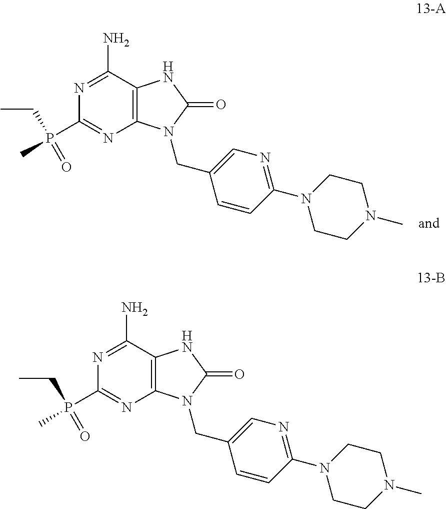

### Step 1: Preparation of 1-methylphosphonoyloxyethane (Compound 13a)

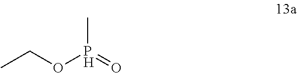

To diethoxy(methyl)phosphane (60.0 g, 440.76 mmol, 1 eq) was added H₂O (7.94 g, 440.76 mmol, 1 eq) at 0° C. Then the mixture was stirred at 25° C. for 12 hrs. ¹H NMR showed the reaction was complete and the desired product was obtained, which containing 50 mol % ethanol. The reaction solution was used into next step directly. ¹H NMR (400 MHz, CDCl₃) δ ppm 7.90-7.88 (m, 0.5H), 6.55-6.54 (m, 0.5H), 4.21-4.06 (m, 2H), 1.56 (dd, J=14.8, 2.0 Hz, 3H), 1.37 (t, J=3.2 Hz, 3H).

### Step 2: Preparation of 1-methylphosphonoylethane (Compound 13b)

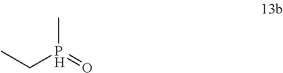

Ethylmagnesium bromide (440 mL, 3M, 1321 mmol, 3 eq) was added to a solution of 1-methylphosphonoyloxyethane (47.6 g, Compound 13a, 440.41 mmol, 1 eq) in THF (900 mL) dropwise over 30 min at 0° C. under N₂. Then the mixture was warmed to 25° C. and stirred for 12 hrs. The reaction mixture was cooled to 0° C. A solution of potassium carbonate (182.6 g, 1321 mmol, 3 eq) in water (200 mL) was added dropwise to the reaction mixture. The mixture was extracted with DCM (3×800 mL). Then the organic layers were combined and dried over Na₂SO₄. After filtration, the filtrate was concentrated in vacuo to afford 1-methylphosphonoylethane (47 g, Compound 13b, crude) as a light yellow liquid. ¹H NMR (400 MHz, CDCl₃) δ ppm 7.53-7.50 (m, 0.5H), 6.40-6.37 (m, 0.5H), 1.85-1.82 (m, 2H), 1.57 (dd, J=14.8, 2.0 Hz, 3H), 1.24-1.15 (m, 3H).

### Step 3: Preparation of 2-[ethyl(methyl)phosphoryl]-N,9-bis[(4-methoxyphenyl)methyl]purin-6-amine (Compound 13c)

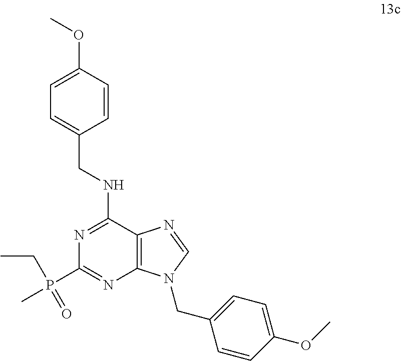 [SMILES: CCP(C)(=O)c1nc(NCc2ccc(OC)cc2)c2ncn(Cc3ccc(OC)cc3)c2n1]

The title compound was prepared in analogy to Example 1, Step 3 by using 1-methylphosphonoylethane (Compound 13b) instead of methylphosphonoylmethane. 2-[Ethyl(methyl)phosphoryl]-N,9-bis[(4-methoxyphenyl)methyl]purin-6-amine (78 g, Compound 13c, crude) was obtained as a black oil and used in next step without further purification. MS obsd. (ESI⁺) [(M+H)⁺]: 466.2.

### Step 4: Preparation of 2-[ethyl(methyl)phosphoryl]-9H-purin-6-amine (Compound 13d)

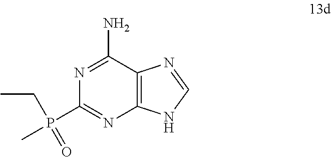

The title compound was prepared in analogy to Example 1, Step 4 by using 2-[ethyl(methyl)phosphoryl]-N,9-bis[(4-methoxyphenyl)methyl]purin-6-amine (Compound 13c) instead of 2-dimethylphosphoryl-N,9-bis[(4-methoxyphenyl)methyl]purin-6-amine (Compound 1c). 2-[Ethyl(methyl)phosphoryl]-9H-purin-6-amine (37 g, Compound 13d) was obtained as a black oil and used in next step without further purification. MS obsd. (ESI⁺) [(M+H)⁺]: 226.0.

### Step 5: Preparation of 9-[(6-chloro-3-pyridyl)methyl]-2-[ethyl(methyl)phosphoryl]purin-6-amine (Compound 13e)

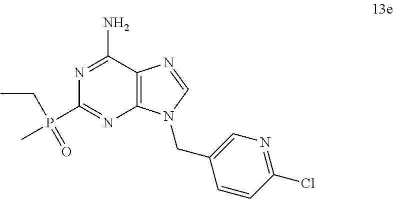 [SMILES: CCP(C)(=O)c1nc(N)c2ncn(Cc3ccc(Cl)nc3)c2n1]

The title compound was prepared in analogy to Example 1, Step 5 by using 2-[ethyl(methyl)phosphoryl]-9H-purin-6-amine (Compound 13d) instead of 2-dimethylphosphoryl-9H-purin-6-amine (Compound 1d). 9-[(6-Chloro-3-pyridyl)methyl]-2-[ethyl(methyl)phosphoryl]purin-6-amine (30 g, Compound 13e) was obtained as a light yellow solid. MS obsd. (ESI⁺) [{³⁵Cl}(M+H)⁺]: 351.0. ¹H NMR (400 MHz, DMSO-d₆) δ ppm 8.54 (d, J=2.4 Hz, 1H), 8.48 (s, 1H), 7.88 (dd, J=8.0, 2.4 Hz, 1H), 7.50 (d, J=8.0 Hz, 1H), 5.46 (s, 2H), 2.06-1.97 (m, 2H), 1.69 (d, J=13.2 Hz, 3H), 1.01 (dt, J=17.6, 7.6 Hz, 3H).

### Step 6: Preparation of 8-bromo-9-[(6-chloro-3-pyridyl)methyl]-2-[(R)-ethyl(methyl)phosphoryl]purin-6-amine (Compound 13f-A) and 8-bromo-9-[(6-chloro-3-pyridyl)methyl]-2-[(S)-ethyl(methyl)phosphoryl]purin-6-amine (Compound 13f-B)

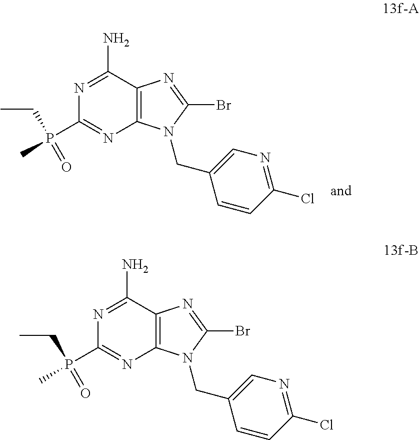 [SMILES: CC[P@@](C)(=O)c1nc(N)c2nc(Br)n(Cc3ccc(Cl)nc3)c2n1 ; CC[P@](C)(=O)c1nc(N)c2nc(Br)n(Cc3ccc(Cl)nc3)c2n1]

The title compounds were prepared in analogy to Example 1, Step 6 by using 9-[(6-chloro-3-pyridyl)methyl]-2-[ethyl(methyl)phosphoryl]purin-6-amine (Compound 13e) instead of 9-[(6-chloro-3-pyridyl)methyl]-2-dimethylphosphoryl-purin-6-amine (Compound 1e). After prep-HPLC purification, the racemic product was separated by chiral SFC to afford 8-bromo-9-[(6-chloro-3-pyridyl)methyl]-2-[(R)-ethyl(methyl)phosphoryl]purin-6-amine (faster eluting, 15 g, Compound 13f-A) as a brown solid and 8-bromo-9-[(6-chloro-3-pyridyl)methyl]-2-[(S)-ethyl(methyl)phosphoryl]purin-6-amine (slower eluting, 15.7 g, Compound 13f-B) as a brown solid. Chiral SFC condition: DAICEL CHIRALPAK AD (10 μm, 50×250 mm) column; Mobile phase: Phase A for CO₂, and Phase B for EtOH (0.05% DEA); Gradient elution: EtOH (0.05% DEA) in CO₂ from 5% to 40%; Flow rate: 3 mL/min; Detector: PDA.

Compound 13f-A: MS obsd. (ESI⁺) [{³⁵Cl&⁷⁹Br}(M+H)⁺]: 430.9. ¹H NMR (400 MHz, DMSO-d₆) δ ppm 8.47 (d, J=2.4 Hz, 1H), 7.77 (br s, 2H), 7.73 (dd, J=8.0, 2.4 Hz, 1H), 7.52 (d, J=8.0 Hz, 1H), 5.44 (s, 2H), 2.04-1.93 (m, 2H), 1.65 (d, J=13.2 Hz, 3H), 1.01 (dt, J=17.6, 7.6 Hz, 3H).

Compound 13f-B: MS obsd. (ESI⁺) [{³⁵Cl&⁷⁹Br}(M+H)⁺]: 430.9. ¹H NMR (400 MHz, DMSO-d₆) δ ppm 8.47 (d, J=2.4 Hz, 1H), 7.77 (br s, 2H), 7.73 (dd, J=8.0, 2.4 Hz, 1H), 7.51 (d, J=8.4 Hz, 1H), 5.44 (s, 2H), 2.04-1.93 (m, 2H), 1.64 (d, J=13.2 Hz, 3H), 1.02 (dt, J=17.6, 7.6 Hz, 3H).

### Step 7: Preparation of 6-amino-9-[(6-chloro-3-pyridyl)methyl]-2-[(R)-ethyl(methyl)phosphoryl]-7H-purin-8-one (Compound 13g-A) and 6-amino-9-[(6-chloro-3-pyridyl)methyl]-2-[(S)-ethyl(methyl)phosphoryl]-7H-purin-8-one (Compound 13g-B)

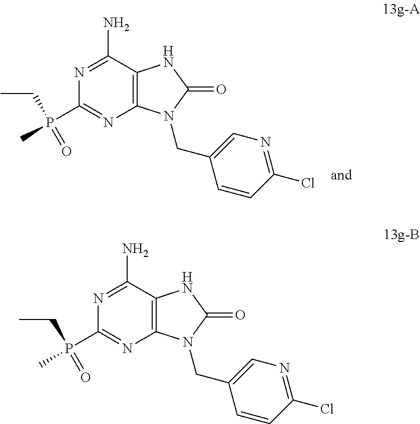 [SMILES: CC[P@@](C)(=O)c1nc(N)c2[nH]c(=O)n(Cc3ccc(Cl)nc3)c2n1 ; CC[P@](C)(=O)c1nc(N)c2[nH]c(=O)n(Cc3ccc(Cl)nc3)c2n1]

Compound 13g-A was prepared in analogy to Example 1, Step 7 by using 8-bromo-9-[(6-chloro-3-pyridyl)methyl]-2-[(R)-ethyl(methyl)phosphoryl]purin-6-amine (Compound 13f-A) instead of 8-bromo-9-[(6-chloro-3-pyridyl)methyl]-2-dimethylphosphoryl-purin-6-amine (Compound 1f). 6-Amino-9-[(6-chloro-3-pyridyl)methyl]-2-[(R)-ethyl(methyl)phosphoryl]-7H-purin-8-one (11 g, Compound 13g-A) was obtained as a grey solid. MS obsd. (ESI⁺) [{³⁵Cl}(M+H)⁺]: 367.0. ¹H NMR (400 MHz, DMSO-d₆) δ ppm 8.43 (d, J=2.0 Hz, 1H), 7.77 (dd, J=8.4, 2.4 Hz, 1H), 7.46 (d, J=8.4 Hz, 1H), 7.09 (br s, 2H), 4.99 (s, 2H), 2.00-1.88 (m, 2H), 1.59 (d, J=13.2 Hz, 3H), 1.00 (dt, J=17.6, 7.6 Hz, 3H).

Compound 13g-B was prepared in analogy to Compound 13g-A by using 8-bromo-9-[(6-chloro-3-pyridyl)methyl]-2-[(S)-ethyl(methyl)phosphoryl]purin-6-amine (Compound 13f-B) instead of 8-bromo-9-[(6-chloro-3-pyridyl)methyl]-2-[(R)-ethyl(methyl)phosphoryl]purin-6-amine (Compound 13f-A). 6-Amino-9-[(6-chloro-3-pyridyl)methyl]-2-[(S)-ethyl(methyl)phosphoryl]-7H-purin-8-one (11.5 g, Compound 13g-B) was obtained as a grey solid. MS obsd. (ESI⁺) [{³⁵Cl}(M+H)⁺]: 367.0. ¹H NMR (400 MHz, DMSO-d₆) δ ppm 8.42 (d, J=2.4 Hz, 1H), 7.77 (dd, J=8.4, 2.4 Hz 1H), 7.46 (d, J=8.4 Hz, 1H), 6.98 (br s, 2H), 4.98 (s, 2H), 1.99-1.88 (m, 2H), 1.58 (d, J=13.2 Hz, 3H), 0.99 (dt, J=17.6, 7.6 Hz, 3H).

### Step 8: Preparation of 6-amino-2-[(R)-ethyl(methyl)phosphoryl]-9-[[6-(4-methylpiperazin-1-yl)-3-pyridyl]methyl]-7H-purin-8-one (Example 13-A) and 6-amino-2-[(S)-ethyl(methyl)phosphoryl]-9-[[6-(4-methylpiperazin-1-yl)-3-pyridyl]methyl]-7H-purin-8-one

### Example 13-B

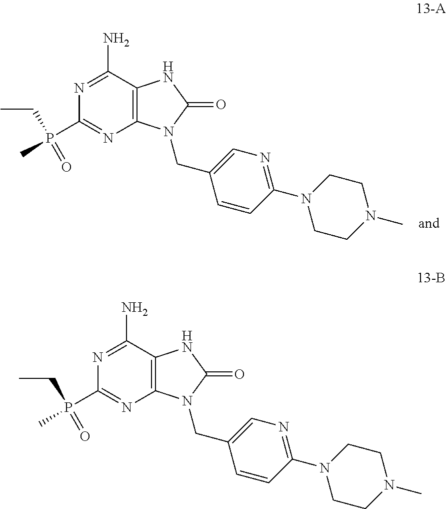

A solution of 6-amino-9-[(6-chloro-3-pyridyl)methyl]-2-[(R)-ethyl(methyl)phosphoryl]-7H-purin-8-one (300.0 mg, Compound 13g-A, 0.820 mmol, 1 eq) in 1-methylpiperazine (8.0 mL, 40.9 mmol, 50 eq) was stirred at 170° C. for 6 hrs. The solution was purified by prep-HPLC. The residue was purified by SFC (DAICEL CHIRALCEL OD (10 μm, 30×250 mm) column; mobile phase: [0.1% NH₃H₂O EtOH]; B %: 40%-40%, 3.2 min; 25 min) to afford 6-amino-2-[(R)-ethyl(methyl)phosphoryl]-9-[[6-(4-methylpiperazin-1-yl)-3-pyridyl]methyl]-7H-purin-8-one (108.3 mg, Example 13-A) as a white solid. MS obsd. (ESI⁺) [(M+H)⁺]: 431.2. ¹H NMR (400 MHz, DMSO-d₆) δ ppm 10.41 (s, 1H), 8.15 (d, J=2.4 Hz, 1H), 7.54 (dd, J=8.8, 2.4 Hz, 1H), 6.78 (d, J=8.8 Hz, 1H), 6.67 (br s, 2H), 4.81 (s, 2H), 3.43 (t, J=4.8 Hz, 4H), 2.37 (t, J=4.8 Hz, 4H), 2.21 (s, 3H), 2.05-1.98 (m, 2H), 1.63 (d, J=13.2 Hz, 3H), 1.04 (dt, J=17.6, 7.6 Hz, 3H).

Example 13-B was prepared in analogy to Example 13-A by using 6-amino-9-[(6-chloro-3-pyridyl)methyl]-2-[(S)-ethyl(methyl)phosphoryl]-7H-purin-8-one (Compound 13g-B) instead of 6-amino-9-[(6-chloro-3-pyridyl)methyl]-2-[(R)-ethyl(methyl)phosphoryl]-7H-purin-8-one (Compound 13g-A). 6-Amino-2-[(S)-ethyl(methyl)phosphoryl]-9-[[6-(4-methylpiperazin-1-yl)-3-pyridyl]methyl]-7H-purin-8-one (119.3 mg, Example 13-B) was obtained as a white solid. MS obsd. (ESI⁺) [(M+H)⁺]: 431.2. ¹H NMR (400 MHz, DMSO-d₆) δ ppm 10.41 (s, 1H), 8.14 (d, J=2.4 Hz, 1H), 7.53 (dd, J=8.8, 2.4 Hz, 1H), 6.77 (d, J=8.8 Hz, 1H), 6.67 (br s, 2H), 4.80 (s, 2H), 3.42 (t, J=4.8 Hz, 4H), 2.34 (t, J=4.8 Hz, 4H), 2.18 (s, 3H), 2.05-1.90 (m, 2H), 1.62 (d, J=12.8 Hz, 3H), 1.03 (dt, J=17.6, 7.6 Hz, 3H).

### Example 14-A and Example 14-B

### 6-amino-2-[(R)-ethyl(methyl)phosphoryl]-9-[[6-(4-methylsulfonylpiperazin-1-yl)-3-pyridyl]methyl]-7H-purin-8-one (Example 14-A) and 6-amino-2-[(S)-ethyl(methyl)phosphoryl]-9-[[6-(4-methylsulfonylpiperazin-1-yl)-3-pyridyl]methyl]-7H-purin-8-one (Example 14-B)

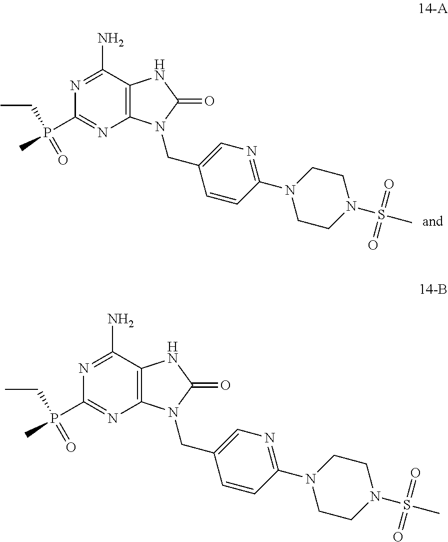

A solution of 6-amino-9-[(6-chloro-3-pyridyl)methyl]-2-[(R)-ethyl(methyl)phosphoryl]-7H-purin-8-one (250.0 mg, Compound 13g-A, 0.680 mmol, 1 eq) and 1-methylsulfonylpiperazine (1.1 g, 6.82 mmol, 10 eq) in THF (2 mL) was heated to 170° C. The solvent was evaporated and the residue was stirred at 170° C. for 8 hrs. The mixture was purified by reverse flash chromatography and prep-HPLC to afford 6-amino-2-[(R)-ethyl(methyl)phosphoryl]-9-[[6-(4-methylsulfonylpiperazin-1-yl)-3-pyridyl]methyl]-7H-purin-8-one (19.8 mg, Example 14-A) as a white solid. MS obsd. (ESI⁺) [(M+H)⁺]: 495.4. ¹H NMR (400 MHz, DMSO-d₆) δ ppm 10.63 (br s, 1H), 8.19 (t, J=2.4 Hz, 1H), 7.60 (dd, J=8.8, 2.4 Hz, 1H), 6.86 (d, J=8.8 Hz, 1H), 6.71 (br s, 2H), 4.83 (s, 2H), 3.59 (t, J=4.8 Hz, 4H), 3.15 (t, J=4.8 Hz, 4H), 2.88 (s, 3H), 2.06-1.88 (m, 2H), 1.65 (d, J=13.2 Hz, 3H), 1.04 (dt, J=17.2, 7.6 Hz, 3H).

Example 14-B was prepared in analogy to Example 14-A by using 6-amino-9-[(6-chloro-3-pyridyl)methyl]-2-[(S)-ethyl(methyl)phosphoryl]-7H-purin-8-one (Compound 13g-B) instead of 6-amino-9-[(6-chloro-3-pyridyl)methyl]-2-[(R)-ethyl(methyl)phosphoryl]-7H-purin-8-one (Compound 13g-A). 6-Amino-2-[(S)-ethyl(methyl)phosphoryl]-9-[[6-(4-methylsulfonylpiperazin-1-yl)-3-pyridyl]methyl]-7H-purin-8-one (46.5 mg, Example 14-B) was obtained as a white solid. MS obsd. (ESI⁺) [(M+H)⁺]: 495.4. ¹H NMR (400 MHz, DMSO-d₆) δ ppm 10.63 (br s, 1H), 8.18 (t, J=2.4 Hz, 1H), 7.60 (dd, J=8.8, 2.4 Hz, 1H), 6.86 (d, J=8.8 Hz, 1H), 6.62 (br s, 2H), 4.81 (s, 2H), 3.59 (t, J=4.8 Hz, 4H), 3.15 (t, J=4.8 Hz, 4H), 2.87 (s, 3H), 2.03-1.85 (m, 2H), 1.64 (d, J=13.2 Hz, 3H), 1.04 (dt, J=17.2, 7.6 Hz, 3H).

### Example 15-A and Example 15-B

### 6-amino-9-[[6-[2-(dimethylamino)ethylamino]-3-pyridyl]methyl]-2-[(R)-ethyl(methyl)phosphoryl]-7H-purin-8-one (Example 15-A) and 6-amino-9-[[6-[2-(dimethylamino)ethylamino]-3-pyridyl]methyl]-2-[(S)-ethyl(methyl)phosphoryl]-7H-purin-8-one (Example 15-B)

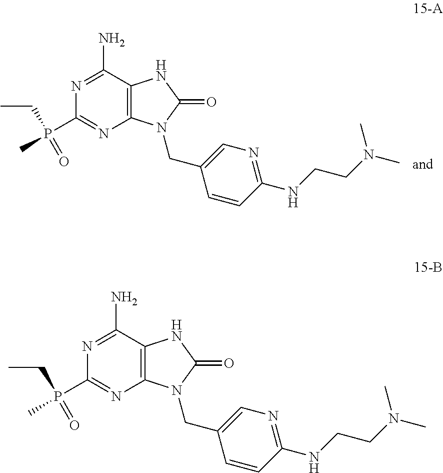

Example 15-A was prepared in analogy to Example 1, Step 8 by using 6-amino-9-[(6-chloro-3-pyridyl)methyl]-2-[(R)-ethyl(methyl)phosphoryl]-7H-purin-8-one (Compound 13g-A) instead of 6-amino-9-[(6-chloro-3-pyridyl)methyl]-2-dimethylphosphoryl-7H-purin-8-one (Compound 1g). 6-Amino-9-[[6-[2-(dimethylamino)ethylamino]-3-pyridyl]methyl]-2-[(R)-ethyl(methyl)phosphoryl]-7H-purin-8-one (1.1 g, Example 15-A) was obtained as a white solid. MS obsd. (ESI⁺) [(M+H)⁺]: 419.2. ¹H NMR (400 MHz, DMSO-d₆) δ ppm 10.46 (br s, 1H), 8.01 (d, J=2.0 Hz, 1H), 7.39 (dd, J=8.8, 2.4 Hz, 1H), 6.69 (br s, 2H), 6.41 (d, J=8.8 Hz, 1H), 6.36 (t, J=5.6 Hz, 1H), 4.74 (s, 2H), 3.27 (q, J=6.4 Hz, 2H), 2.35 (t, J=6.4 Hz, 2H), 2.14 (s, 6H), 2.08-1.92 (m, 2H), 1.65 (d, J=13.2 Hz, 3H), 1.03 (dt, J=17.6, 7.6 Hz, 3H).

Example 15-B was prepared in analogy to Example 15-A by using 6-amino-9-[(6-chloro-3-pyridyl)methyl]-2-[(S)-ethyl(methyl)phosphoryl]-7H-purin-8-one (Compound 13g-B) instead of 6-amino-9-[(6-chloro-3-pyridyl)methyl]-2-[(R)-ethyl(methyl)phosphoryl]-7H-purin-8-one (Compound 13g-A). 6-Amino-9-[[6-[2-(dimethylamino)ethylamino]-3-pyridyl]methyl]-2-[(S)-ethyl(methyl)phosphoryl]-7H-purin-8-one (2.2 g, Example 15-B) was obtained as a white solid. MS obsd. (ESI⁺) [(M+H)⁺]: 419.2. ¹H NMR (400 MHz, DMSO-d₆) δ ppm 10.46 (br s, 1H), 8.01 (d, J=2.0 Hz, 1H), 7.39 (dd, J=8.8, 2.4 Hz, 1H), 7.02 (br s, 2H), 6.39 (d, J=8.8 Hz, 1H), 6.26 (t, J=5.6 Hz, 1H), 4.75 (s, 2H), 3.25 (q, J=6.4 Hz, 2H), 2.34 (t, J=6.4 Hz, 2H), 2.13 (s, 6H), 2.12-1.95 (m, 2H), 1.62 (d, J=13.2 Hz, 3H), 1.03 (dt, J=17.6, 7.6 Hz, 3H). The configuration of the stereo center of Example 15-B was confirmed by X-ray crystallographic analysis.

### Example 16-A and Example 16-B

### 6-amino-9-[[6-[2-(dimethylamino)ethoxy]-3-pyridyl]methyl]-2-[(R)-ethyl(methyl)phosphoryl]-7H-purin-8-one (Example 16-A) and 6-amino-9-[[6-[2-(dimethylamino)ethoxy]-3-pyridyl]methyl]-2-[(S)-ethyl(methyl)phosphoryl]-7H-purin-8-one (Example 16-B)

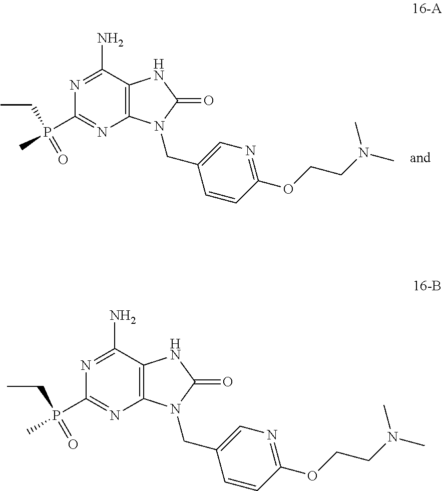

Example 16-A was prepared in analogy to Example 2 by using 6-amino-9-[(6-chloro-3-pyridyl)methyl]-2-[(R)-ethyl(methyl)phosphoryl]-7H-purin-8-one (Compound 13g-A) instead of 6-amino-9-[(6-chloro-3-pyridyl)methyl]-2-dimethylphosphoryl-7H-purin-8-one (Compound 1g). 6-Amino-9-[[6-[2-(dimethylamino)ethoxy]-3-pyridyl]methyl]-2-[(R)-ethyl(methyl)phosphoryl]-7H-purin-8-one (6.1 mg, Example 16-A) was obtained as a white solid. MS obsd. (ESI⁺) [(M+H)⁺]: 418.1. ¹H NMR (400 MHz, CD₃OD) a ppm 8.25 (d, J=2.4 Hz, 1H), 7.79 (dd, J=8.8, 2.4 Hz, 1H), 6.76 (d, J=8.4 Hz, 1H), 5.02 (s, 2H), 4.39 (t, J=5.6 Hz, 2H), 2.74 (t, J=5.6 Hz, 2H), 2.31 (s, 6H), 2.20-2.11 (m, 2H), 1.80 (d, J=13.2 Hz, 3H), 1.16 (dt, J=18.0, 7.6 Hz, 3H).

Example 16-B was prepared in analogy to Example 16-A by using 6-amino-9-[(6-chloro-3-pyridyl)methyl]-2-[(S)-ethyl(methyl)phosphoryl]-7H-purin-8-one (Compound 13g-B) instead of 6-amino-9-[(6-chloro-3-pyridyl)methyl]-2-[(R)-ethyl(methyl)phosphoryl]-7H-purin-8-one (Compound 13g-A). 6-Amino-9-[[6-[2-(dimethylamino)ethoxy]-3-pyridyl]methyl]-2-[(S)-ethyl(methyl)phosphoryl]-7H-purin-8-one (7.8 mg, Example 16-B) was obtained as a white solid. MS obsd. (ESI⁺) [(M+H)⁺]: 418.1. ¹H NMR (400 MHz, DMSO-d₆) δ ppm 10.45 (br s, 1H), 8.18 (d, J=2.4 Hz, 1H), 7.68 (dd, J=8.4, 2.4 Hz, 1H), 6.76 (d, J=8.4 Hz, 1H), 6.70 (s, 2H), 4.89 (s, 2H), 4.30 (t, J=5.6 Hz, 2H), 2.57 (t, J=5.6 Hz, 2H), 2.17 (s, 6H), 2.05-1.86 (m, 2H), 1.62 (d, J=13.2 Hz, 3H), 1.02 (dt, J=18.0, 7.6 Hz, 3H).

### Example 17

### 6-amino-2-[(R)-ethyl(methyl)phosphoryl]-9-[[6-[2-(methylamino)ethylamino]-3-pyridyl]methyl]-7H-purin-8-one

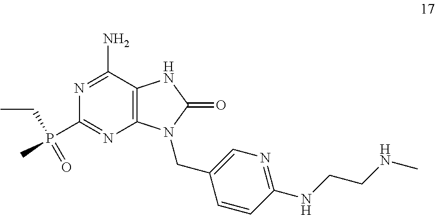 [SMILES: CC[P@@](C)(=O)c1nc(N)c2[nH]c(=O)n(Cc3ccc(NCCNC)nc3)c2n1]

### Step 1: Preparation of tert-butyl N-[2-[[5-[[6-amino-2-[(R)-ethyl(methyl)phosphoryl]-8-oxo-7H-purin-9-yl]methyl]-2-pyridyl]amino]ethyl]-N-methyl-carbamate (Compound 17a)

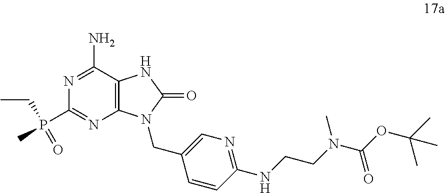 [SMILES: CC[P@@](C)(=O)c1nc(N)c2[nH]c(=O)n(Cc3ccc(NCCN(C)C(=O)OC(C)(C)C)nc3)c2n1]

The title compound was prepared in analogy to Example 1, Step 8 by using 6-amino-9-[(6-chloro-3-pyridyl)methyl]-2-[(R)-ethyl(methyl)phosphoryl]-7H-purin-8-one (Compound 13g-A) instead of 6-amino-9-[(6-chloro-3-pyridyl)methyl]-2-dimethylphosphoryl-7H-purin-8-one (Compound 1g), and tert-butyl N-(2-aminoethyl)-N-methyl-carbamate instead of N′,N′-dimethylethane-1,2-diamine. tert-Butyl N-[2-[[5-[[6-amino-2-[(R)-ethyl(methyl)phosphoryl]-8-oxo-7H-purin-9-yl]methyl]-2-pyridyl]amino]ethyl]-N-methyl-carbamate (50 mg, Compound 17a) was obtained as a white solid. MS obsd. (ESI⁺) [(M+H)⁺]: 505.2.

### Step 2: Preparation of 6-amino-2-[(R)-ethyl(methyl)phosphoryl]-9-[[6-[2-(methylamino)ethylamino]-3-pyridyl]methyl]-7H-purin-8-one (Example 17)

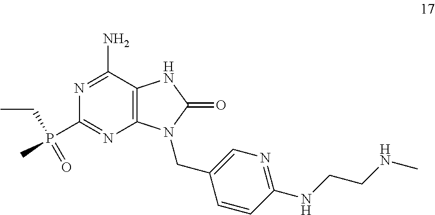 [SMILES: CC[P@@](C)(=O)c1nc(N)c2[nH]c(=O)n(Cc3ccc(NCCNC)nc3)c2n1]

To a solution of tert-butyl N-[2-[[5-[[6-amino-2-[(R)-ethyl(methyl)phosphoryl]-8-oxo-7H-purin-9-yl]methyl]-2-pyridyl]amino]ethyl]-N-methyl-carbamate (30.0 mg, Compound 17a, 0.060 mmol, 1 eq) in DCM (2 mL) was added TFA (0.5 mL). The reaction mixture was stirred at 20° C. for 2 h, then concentrated in vacuo and the residue was purified by prep-HPLC to afford 6-amino-2-[(R)-ethyl(methyl)phosphoryl]-9-[[6-[2-(methylamino)ethylamino]-3-pyridyl]methyl]-7H-purin-8-one (7.2 mg, Example 17) as a white solid. MS obsd. (ESI⁺) [(M+H)⁺]: 405.2. ¹H NMR (400 MHz, CD₃OD) δ ppm 8.26 (d, J=2.0 Hz, 1H), 7.73 (dd, J=8.4, 2.0 Hz, 1H), 6.67 (d, J=8.8 Hz, 1H), 4.97 (s, 2H), 3.81 (t, J=6.0 Hz, 2H), 3.16 (t, J=6.0 Hz, 2H), 3.04 (s, 3H), 2.21-2.12 (m, 2H), 1.81 (d, J=13.6 Hz, 3H), 1.18 (dt, J=18.0, 7.6 Hz, 3H).

### Example 18-A and Example 18-B

### 6-amino-9-[[6-[2-(dimethylamino)ethylamino]-3-pyridyl]methyl]-2-[(S)-methyl(propyl)phosphoryl]-7H-purin-8-one (Example 18-A) and 6-amino-9-[[6-[2-(dimethylamino)ethylamino]-3-pyridyl]methyl]-2-[(R)-methyl(propyl)phosphoryl]-7H-purin-8-one (Example 18-B)

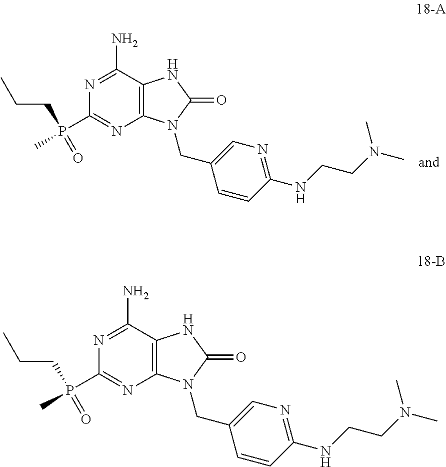

### Step 1: Preparation of 1-methylphosphonoylpropane (Compound 18a)

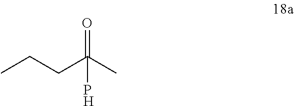

The title compound was prepared in analogy to Example 13, Step 2 by using n-propylmagnesium bromide instead of ethylmagnesium bromide. 1-Methylphosphonoylpropane (crude, 16 g, Compound 18a) was obtained as a light yellow liquid. ¹H NMR (400 MHz, CDCl₃) δ ppm: 7.63-7.54 (m, 0.5H), 6.48-6.41 (m, 0.5H), 1.89-1.64 (m, 4H), 1.56 (dd, J=13.6, 4.0 Hz, 3H), 1.07 (t, J=6.8 Hz, 3H).

### Step 2: Preparation of N,9-bis[(4-methoxyphenyl)methyl]-2-[methyl(propyl)phosphoryl]purin-6-amine (Compound 18b)

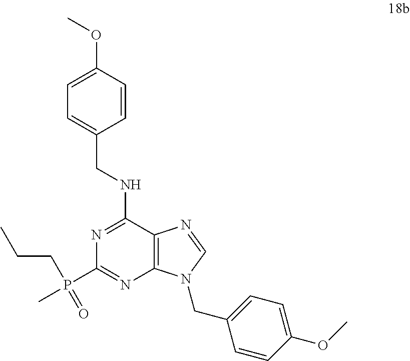 [SMILES: CCCP(C)(=O)c1nc(NCc2ccc(OC)cc2)c2ncn(Cc3ccc(OC)cc3)c2n1]

The title compound was prepared in analogy to Example 1, Step 3 by using 1-methylphosphonoylpropane (Compound 18a) instead of methylphosphonoylmethane. N,9-bis[(4-methoxyphenyl)methyl]-2-[methyl(propyl)phosphoryl]purin-6-amine (crude, 35 g, Compound 18b) was obtained as a black oil which was used in the next step without further purification. MS obsd. (ESI⁺) [(M+H)⁺]: 480.1.

### Step 3: Preparation of 2-[methyl(propyl)phosphoryl]-9H-purin-6-amine (Compound 18c)

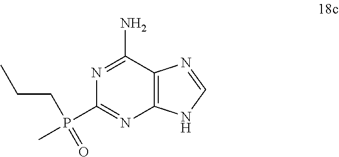 [SMILES: CCCP(C)(=O)c1nc(N)c2nc[nH]c2n1]

The title compound was prepared in analogy to Example 1, Step 4 by using N,9-bis[(4-methoxyphenyl)methyl]-2-[methyl(propyl)phosphoryl]purin-6-amine (Compound 18b) instead of 2-dimethylphosphoryl-N,9-bis[(4-methoxyphenyl)methyl]purin-6-amine (Compound 1c). 2-[Methyl(propyl)phosphoryl]-9H-purin-6-amine (crude, 16 g, Compound 18c) was obtained as a red oil which was used in the next step without further purification. MS obsd. (ESI⁺) [(M+H)⁺]: 240.0.

### Step 4: Preparation of 9-[(6-chloro-3-pyridyl)methyl]-2-[methyl(propyl)phosphoryl]purin-6-amine (Compound 18d)

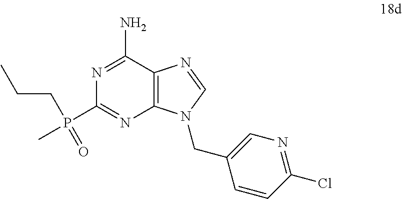 [SMILES: CCCP(C)(=O)c1nc(N)c2ncn(Cc3ccc(Cl)nc3)c2n1]

The title compound was prepared in analogy to Example 1, Step 5 by using 2-[methyl(propyl)phosphoryl]-9H-purin-6-amine (Compound 18c) instead of 2-dimethylphosphoryl-9H-purin-6-amine (Compound 1d). 9-[(6-Chloro-3-pyridyl)methyl]-2-[methyl(propyl)phosphoryl]purin-6-amine (15 g, Compound 18d) was obtained as a light yellow solid. MS obsd. (ESI⁺) [(M+H)⁺]: 365.1. ¹H NMR (400 MHz, DMSO-d₆) δ ppm: 8.53-8.52 (m, 2H), 7.87 (dd, J=8.4, 2.4 Hz, 1H), 7.50 (d, J=8.4 Hz, 1H), 5.49 (s, 2H), 2.06-1.93 (m, 2H), 1.71 (d, J=13.2 Hz, 3H), 1.51-1.40 (m, 2H), 0.92 (t, J=7.6 Hz, 3H).

### Step 5: Preparation of 8-bromo-9-[(6-chloro-3-pyridyl)methyl]-2-[(S)-methyl(propyl)phosphoryl]purin-6-amine (Compound 18e-A) and 8-bromo-9-[(6-chloro-3-pyridyl)methyl]-2-[(R)-methyl(propyl)phosphoryl]purin-6-amine (Compound 18e-B)

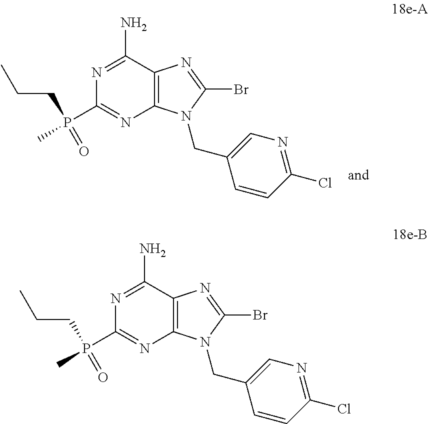 [SMILES: CCC[P@](C)(=O)c1nc(N)c2nc(Br)n(Cc3ccc(Cl)nc3)c2n1 ; CCC[P@@](C)(=O)c1nc(N)c2nc(Br)n(Cc3ccc(Cl)nc3)c2n1]

The title compounds were prepared in analogy to Example 1, Step 6 by using 9-[(6-chloro-3-pyridyl)methyl]-2-[methyl(propyl)phosphoryl]purin-6-amine (Compound 18d) instead of 9-[(6-chloro-3-pyridyl)methyl]-2-dimethylphosphoryl-purin-6-amine (Compound 1e). After prep-HPLC purification, the racemic product was separated by chiral SFC to afford 8-bromo-9-[(6-chloro-3-pyridyl)methyl]-2-[(S)-methyl(propyl)phosphoryl]purin-6-amine (faster eluting, 6.0 g, Compound 18e-A) as a brown solid and 8-bromo-9-[(6-chloro-3-pyridyl)methyl]-2-[(R)-methyl(propyl)phosphoryl]purin-6-amine (slower eluting, 6.0 g, Compound 18e-B) as a brown solid. Chiral SFC condition: DAICEL CHIRALPAK AD (10 μm, 50×250 mm) column; Mobile phase: Phase A for CO₂, and Phase B for iPrOH (0.1% NH₃H₂O); Gradient elution: iPrOH (0.1% NH₃H₂O) in CO₂ 40%-40%; Flow rate: 3 mL/min; Detector: PDA.

Compound 18e-A: MS obsd. (ESI⁺) [{³⁵Cl&⁷⁹Br}(M+H)⁺]: 444.9. ¹H NMR (400 MHz, DMSO-d₆) δ ppm 8.46 (s, 1H), 7.81-7.74 (m, 2H), 7.72 (dd, J=8.4, 2.4 Hz, 1H), 7.51 (d, J=8.4 Hz, 1H), 5.44 (s, 2H), 2.10-1.85 (m, 2H), 1.65 (d, J=13.2 Hz, 3H), 1.54-1.42 (m, 2H), 0.93 (t, J=7.2 Hz, 3H). The configuration of the stereo center of Compound 18e-A was confirmed by X-ray crystallographic analysis.

Compound 18e-B: MS obsd. (ESI⁺) [{³⁵Cl&⁷⁹Br}(M+H)⁺]: 444.9. ¹H NMR (400 MHz, DMSO-d₆) δ ppm 8.46 (s, 1H), 7.81-7.74 (m, 2H), 7.72 (dd, J=8.4, 2.4 Hz, 1H), 7.51 (d, J=8.4 Hz, 1H), 5.44 (s, 2H), 2.10-1.85 (m, 2H), 1.65 (d, J=13.2 Hz, 3H), 1.54-1.42 (m, 2H), 0.92 (t, J=7.2 Hz, 3H).

### Step 6: Preparation of 6-amino-9-[(6-chloro-3-pyridyl)methyl]-2-[(S)-methyl(propyl)phosphoryl]-7H-purin-8-one (Compound 18f-A) and 6-amino-9-[(6-chloro-3-pyridyl)methyl]-2-[(R)-methyl(propyl)phosphoryl]-7H-purin-8-one (Compound 18f-B)

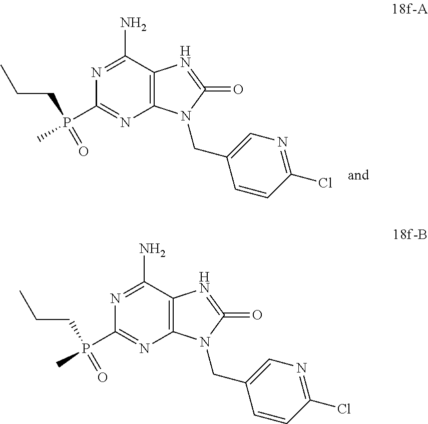

Compound 18f-A was prepared in analogy to Example 1, Step 7 by using 8-bromo-9-[(6-chloro-3-pyridyl)methyl]-2-[(S)-methyl(propyl)phosphoryl]purin-6-amine (Compound 18e-A) instead of 8-bromo-9-[(6-chloro-3-pyridyl)methyl]-2-dimethylphosphoryl-purin-6-amine (Compound 1f). 6-Amino-9-[(6-chloro-3-pyridyl)methyl]-2-[(S)-methyl(propyl)phosphoryl]-7H-purin-8-one (3.6 g, Compound 18f-A) was obtained as a brown solid. MS obsd. (ESI⁺) [{³⁵Cl}(M+H)⁺]: 381.1. ¹H NMR (400 MHz, DMSO-d₆) δ ppm 10.70 (br s, 1H), 8.43 (d, J=2.4 Hz, 1H), 7.78 (dd, J=8.4, 2.4 Hz, 1H), 7.48 (d, J=8.4 Hz, 1H), 6.80 (br s, 2H), 4.99 (s, 2H), 2.00-1.83 (m, 2H), 1.60 (d, J=13.2 Hz, 3H), 1.52-1.38 (m, 2H), 0.92 (t, J=7.2 Hz, 3H).

Compound 18f-B was prepared in analogy to Compound 18f-A by using 8-bromo-9-[(6-chloro-3-pyridyl)methyl]-2-[(R)-methyl(propyl)phosphoryl]purin-6-amine (Compound 18e-B) instead of 8-bromo-9-[(6-chloro-3-pyridyl)methyl]-2-[(S)-methyl(propyl)phosphoryl]purin-6-amine (Compound 18e-A). 6-Amino-9-[(6-chloro-3-pyridyl)methyl]-2-[(R)-methyl(propyl)phosphoryl]-7H-purin-8-one (5.0 g, Compound 18f-B) was obtained as a brown solid. MS obsd. (ESI⁺) [{³⁵Cl}(M+H)⁺]: 381.1. ¹H NMR (400 MHz, DMSO-d₆) δ ppm 10.51 (br s, 1H), 8.43 (d, J=2.4 Hz, 1H), 7.78 (dd, J=8.4, 2.4 Hz, 1H), 7.48 (d, J=8.0 Hz, 1H), 6.73 (br s, 2H), 4.99 (s, 2H), 2.03-1.80 (m, 2H), 1.60 (d, J=13.2 Hz, 3H), 1.53-1.35 (m, 2H), 0.92 (t, J=7.2 Hz, 3H).

### Step 7: Preparation of 6-amino-9-[[6-[2-(dimethylamino)ethylamino]-3-pyridyl]methyl]-2-[(S)-methyl(propyl)phosphoryl]-7H-purin-8-one (Example 18-A) and 6-amino-9-[[6-[2-(dimethylamino)ethylamino]-3-pyridyl]methyl]-2-[(R)-methyl(propyl)phosphoryl]-7H-purin-8-one (Example 18-B)

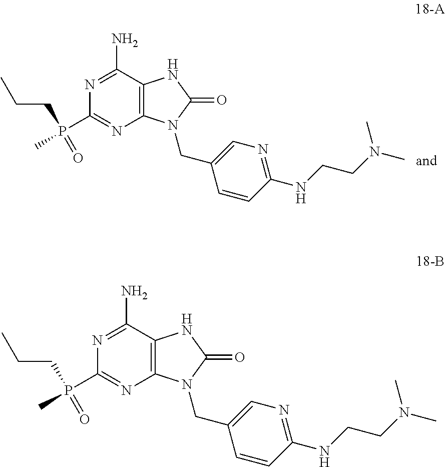

A mixture of 6-amino-9-[(6-chloro-3-pyridyl)methyl]-2-[(S)-methyl(propyl)phosphoryl]-7H-purin-8-one (2.5 g, Compound 18f-A, 6.57 mmol, 1.0 eq) in N′N′-dimethylethane-1,2-diamine (30.0 mL, 32.83 mmol, 5.0 eq) was stirred in a sealed tube at 170° C. for 72 hrs. The reaction mixture was concentrated in vacuo and the residue was purified by prep-HPLC and SFC (DAICEL CHIRALPAK IC (10 μm, 30×250 mm) column; mobile phase: [0.1% NH₃H₂O ⁱPrOH]; B %: 55%-55%, 6.6 min) to afford 6-amino-9-[[6-[2-(dimethylamino)ethylamino]-3-pyridyl]methyl]-2-[(S)-methyl(propyl)phosphoryl]-7H-purin-8-one (1.0 g, Example 18-A) as a white solid. MS obsd. (ESI⁺) [(M+H)⁺]: 433.2. ¹H NMR (400 MHz, DMSO-d₆) δ ppm 8.01 (d, J=2.0 Hz, 1H), 7.38 (dd, J=8.8, 2.4 Hz, 1H), 6.90 (br s, 2H), 6.40 (d, J=8.6 Hz, 1H), 6.32 (t, J=5.4 Hz, 1H), 4.74 (s, 2H), 3.26 (q, J=6.4 Hz, 2H), 2.34 (t, J=6.8 Hz, 2H), 2.13 (s, 6H), 2.04-1.87 (m, 2H), 1.63 (d, J=13.2 Hz, 3H), 1.56-1.42 (m, 2H), 0.96 (t, J=7.2 Hz, 3H).

Example 18-B was prepared in analogy to Example 18-A by using 6-amino-9-[(6-chloro-3-pyridyl)methyl]-2-[(R)-methyl(propyl)phosphoryl]-7H-purin-8-one (Compound 18f-B) instead of 6-amino-9-[(6-chloro-3-pyridyl)methyl]-2-[(S)-methyl(propyl)phosphoryl]-7H-purin-8-one (Compound 18f-A). 6-Amino-9-[[6-[2-(dimethylamino)ethylamino]-3-pyridyl]methyl]-2-[(R)-methyl(propyl)phosphoryl]-7H-purin-8-one (1.0 g, Example 18-B) was obtained as a white solid. MS obsd. (ESI⁺) [(M+H)⁺]: 433.2. ¹H NMR (400 MHz, DMSO-d₆) δ ppm 8.01 (d, J=2.0 Hz, 1H), 7.37 (dd, J=8.8, 2.4 Hz, 1H), 6.68 (br s, 2H), 6.41 (d, J=8.6 Hz, 1H), 6.36 (t, J=5.4 Hz, 1H), 4.74 (s, 2H), 3.28 (q, J=6.4 Hz, 2H), 2.33 (t, J=6.8 Hz, 2H), 2.13 (s, 6H), 2.04-1.87 (m, 2H), 1.63 (d, J=13.2 Hz, 3H), 1.56-1.42 (m, 2H), 0.96 (t, J=7.2 Hz, 3H).

### Example 19

### 6-amino-2-diethylphosphoryl-9-[[6-[2-(dimethylamino)ethylamino]-3-pyridyl]methyl]-7H-purin-8-one

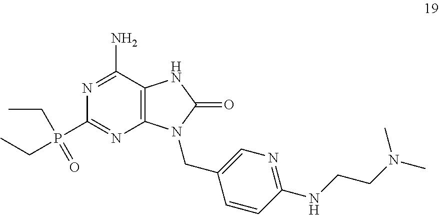 [SMILES: CCP(=O)(CC)c1nc(N)c2[nH]c(=O)n(Cc3ccc(NCCN(C)C)nc3)c2n1]

### Step 1: Preparation of 1-ethylphosphonoylethane (Compound 19a)

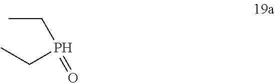

Ethylmagnesium bromide (144.8 mL, 3 M in THF, 434.47 mmol, 3 eq) was added dropwise to a solution of diethyl phosphite (20.0 g, 144.8 mmol, 1 eq) in THF (120 mL) over 30 min at 0° C. under N2. Then the mixture was warmed to 25° C. and stirred for 12 hrs. After the reaction mixture was cooled to 0° C., a solution of potassium carbonate (60.05 g, 434.47 mmol, 3 eq) in water (40 mL) was added dropwise to the stirred solution. The mixture was diluted with DCM (3×50 mL) and filtered. The filtrate was collected, dried over Na₂SO₄, and concentrated under reduced pressure to afford 1-ethylphosphonoylethane (crude, 15.0 g, Compound 19a) as a light yellow liquid. ¹H NMR (400 MHz, CDCl₃) δ ppm 7.43-7.41 (m, 0.5H), 6.29-6.27 (m, 0.5H), 1.93-1.88 (m, 4H), 1.23-1.15 (m, 6H).

### Step 2: Preparation of 2-diethylphosphoryl-N,9-bis[(4-methoxyphenyl)methyl]purin-6-amine (Compound 19b)

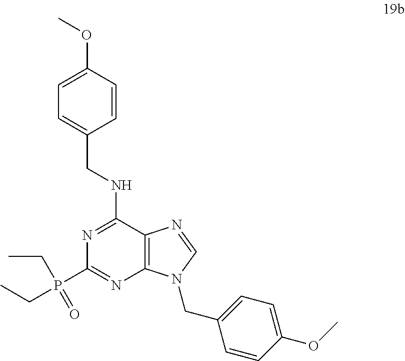 [SMILES: CCP(=O)(CC)c1nc(NCc2ccc(OC)cc2)c2ncn(Cc3ccc(OC)cc3)c2n1]

The title compound was prepared in analogy to Example 1, Step 3 by using 1-ethylphosphonoylethane (Compound 19a) instead of methylphosphonoylmethane. 2-Diethylphosphoryl-N,9-bis[(4-methoxyphenyl)methyl]purin-6-amine (23 g, Compound 19b,) was obtained as a red oil. MS obsd. (ESI⁺) [(M+H)⁺]: 480.2.

### Step 3: Preparation of 2-diethylphosphoryl-9H-purin-6-amine (Compound 19c)

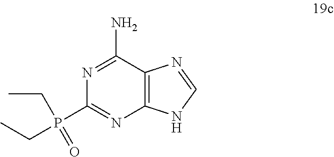 [SMILES: CCP(=O)(CC)c1nc(N)c2nc[nH]c2n1]

The title compound was prepared in analogy to Example 1, Step 4 by using 2-diethylphosphoryl-N,9-bis[(4-methoxyphenyl)methyl]purin-6-amine (Compound 19b) instead of 2-dimethylphosphoryl-N,9-bis[(4-methoxyphenyl)methyl]purin-6-amine (Compound 1c). 2-Diethylphosphoryl-9H-purin-6-amine (crude, 20 g, Compound 19c) was obtained as a red oil. MS obsd. (ESI⁺) [(M+H)⁺]: 240.0.

### Step 4: Preparation of 9-[(6-chloro-3-pyridyl)methyl]-2-diethylphosphoryl-purin-6-amine (Compound 19d)

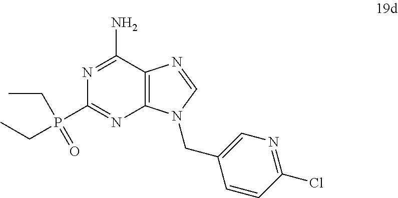 [SMILES: CCP(=O)(CC)c1nc(N)c2ncn(Cc3ccc(Cl)nc3)c2n1]

The title compound was prepared in analogy to Example 1, Step 5 by using 2-diethylphosphoryl-9H-purin-6-amine (Compound 19c) instead of 2-dimethylphosphoryl-9H-purin-6-amine (Compound 1d). 9-[(6-Chloro-3-pyridyl)methyl]-2-diethylphosphoryl-purin-6-amine (20 g, Compound 19d) was obtained as a light yellow oil. MS obsd. (ESI⁺) [{³⁵Cl}(M+H)]: 365.0.

### Step 5: Preparation of 8-bromo-9-[(6-chloro-3-pyridyl)methyl]-2-diethylphosphoryl-purin-6-amine (Compound 19e)

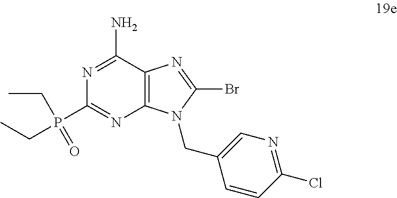 [SMILES: CCP(=O)(CC)c1nc(N)c2nc(Br)n(Cc3ccc(Cl)nc3)c2n1]

The title compound was prepared in analogy to Example 1, Step 6 by using 9-[(6-chloro-3-pyridyl)methyl]-2-diethylphosphoryl-purin-6-amine (Compound 19d) instead of 9-[(6-chloro-3-pyridyl)methyl]-2-dimethylphosphoryl-purin-6-amine (Compound 1e). 8-Bromo-9-[(6-chloro-3-pyridyl)methyl]-2-diethylphosphoryl-purin-6-amine (14 g, Compound 19e) was obtained as a light yellow solid. MS obsd. (ESI⁺) [{³⁵Cl&⁷⁹Br}(M+H)⁺]: 444.9. ¹H NMR (400 MHz, DMSO-d₆) δ ppm 8.46 (d, J=2.4 Hz, 1H), 7.76 (br s, 2H), 7.72 (dd, J=8.4, 2.4 Hz, 1H), 7.52 (d, J=8.0 Hz, 1H), 5.43 (s, 2H), 2.02-1.92 (m, 4H), 1.05-0.97 (m, 6H).

### Step 6: Preparation of 6-amino-9-[(6-chloro-3-pyridyl)methyl]-2-diethylphosphoryl-7H-purin-8-one (Compound 19f)

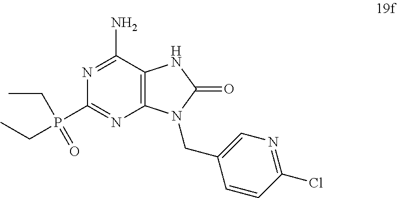 [SMILES: CCP(=O)(CC)c1nc(N)c2[nH]c(=O)n(Cc3ccc(Cl)nc3)c2n1]

The title compound was prepared in analogy to Example 1, Step 7 by using 8-bromo-9-[(6-chloro-3-pyridyl)methyl]-2-diethylphosphoryl-purin-6-amine (Compound 19e) instead of 8-bromo-9-[(6-chloro-3-pyridyl)methyl]-2-dimethylphosphoryl-purin-6-amine (Compound 1f). 6-Amino-9-[(6-chloro-3-pyridyl)methyl]-2-diethylphosphoryl-7H-purin-8-one (11 g, Compound 19f) was obtained as a purple solid. MS obsd. (ESI⁺) [{³⁵Cl}(M+H)⁺]: 381.1. ¹H NMR (400 MHz, DMSO-d₆) δ ppm 8.42 (d, J=2.4 Hz, 1H), 7.79 (dd, J=8.0, 2.4 Hz, 1H), 7.48 (d, J=8.4 Hz, 1H), 6.83 (br s, 2H), 4.98 (s, 2H), 1.97-1.87 (m, 4H), 1.02-0.94 (m, 6H).

### Step 7: Preparation of 6-amino-2-diethylphosphoryl-9-[[6-[2-(dimethylamino)ethylamino]-3-pyridyl]methyl]-7H-purin-8-one (Example 19)

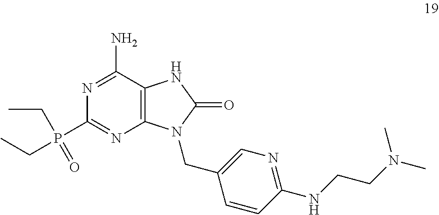 [SMILES: CCP(=O)(CC)c1nc(N)c2[nH]c(=O)n(Cc3ccc(NCCN(C)C)nc3)c2n1]

The title compound was prepared in analogy to Example 1, Step 8 by using 6-amino-9-[(6-chloro-3-pyridyl)methyl]-2-diethylphosphoryl-7H-purin-8-one (Compound 19f) instead of 6-amino-9-[(6-chloro-3-pyridyl)methyl]-2-dimethylphosphoryl-7H-purin-8-one (Compound 1g). 6-Amino-2-diethylphosphoryl-9-[[6-[2-(dimethylamino)ethylamino]-3-pyridyl]methyl]-7H-purin-8-one (3.2 g, Example 19) was obtained as a pink solid. MS obsd. (ESI⁺) [(M+H)⁺]: 433.2. ¹H NMR (400 MHz, DMSO-d₆) δ ppm 8.00 (d, J=2.4 Hz, 1H), 7.38 (dd, J=8.4, 2.4 Hz, 1H), 6.72 (br s, 2H), 6.41 (d, J=8.4 Hz, 1H), 6.36 (t, J=5.6 Hz, 1H), 4.74 (s, 2H), 3.27 (q, J=6.4 Hz, 2H), 2.35 (t, J=6.4 Hz, 2H), 2.14 (s, 6H), 2.14-1.95 (m, 4H), 1.05 (t, J=7.6 Hz, 3H), 1.01 (t, J=7.6 Hz, 3H).

### Reference Compound

Compound RO14 as the Example 14 disclosed in prior art WO 2016180695 was selected as the reference compound for comparison.

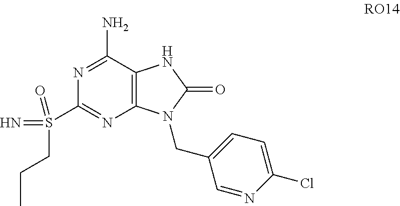 [SMILES: CCCS(=N)(=O)c1nc(N)c2[nH]c(=O)n(Cc3ccc(Cl)nc3)c2n1]

### Example 20

Activity of Compounds of this Invention in HEK293-h TLR7 Assay

### HEK293-Blue-h TLR7 Cells Assay:

A stable HEK293-Blue-h TLR7 cell line was purchased from InvivoGen (Cat. #: hkb-htlr7, San Diego, California, USA). These cells were designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was regulated by the NF-κB promoter upon stimulation of human TLR7 for 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000˜450,000 cells/mL in a volume of 180 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (V/V) heat-inactivated fetal bovine serum for 24 hrs. Then the HEK293-Blue-hTLR7 cells were incubated with addition of 20 μL test compound in a serial dilution in the presence of final DMSO at 1% and perform incubation under 37° C. in a CO₂ incubator for 20 hrs. Then 20 μL of the supernatant from each well was incubated with 180 μL Quanti-blue substrate solution at 37° C. for 2 hours and the absorbance was read at 620˜655 nm using a spectrophotometer. The signalling pathway that TLR7 activation leads to downstream NF-κB activation has been widely accepted, and therefore similar reporter assay was also widely used for evaluating TLR7 agonist (Tsuneyasu Kaisho and Takashi Tanaka, Trends in Immunology, Volume 29, Issue 7, July 2008, Pages 329.sci; Hiroaki Hemmi et al, Nature Immunology 3, 196-200 (2002)).

The compounds of the present invention were tested in HEK293-hTLR7 assay for their TLR7 agonism activity as described herein and results are listed in Table 1. The Examples were found to have EC₅₀ of about 0.001 μM to about 0.08 μM. Particular compounds of the present invention were found to have EC₅₀ of about 0.001 μM which was much better than the reference compound RO14.

TABLE 1
EC₅₀ of the compounds of this invention

| Example No. | HEK293-Blue-h TLR7 (EC₅₀ (μM)) |
|---|---|
| RO14 | 0.017 |
| Example 1 | 0.034 |
| Example 2 | 0.019 |
| Example 3 | 0.028 |
| Example 4 | 0.010 |
| Example 5 | 0.012 |
| Example 6 | 0.027 |
| Example 7 | 0.038 |
| Example 8 | 0.039 |
| Example 9 | 0.021 |
| Example 10 | 0.024 |
| Example 11 | 0.028 |
| Example 12 | 0.084 |
| Example 13-A | 0.006 |
| Example 13-B | 0.005 |
| Example 14-A | 0.008 |
| Example 14-B | 0.007 |
| Example 15-A | 0.018 |
| Example 15-B | 0.012 |
| Example 16-A | 0.001 |
| Example 16-B | 0.006 |
| Example 17 | 0.045 |
| Example 18-A | 0.004 |
| Example 18-B | 0.013 |
| Example 19 | 0.009 |

### Example 21

### Solubility: Lyophilisation Solubility Assay (LYSA) and Thermodynamic Solubility Assay (THESA)

Aqueous solubility is a crucial physicochemical property for any potential drug candidate and is routinely measured in both drug discovery and development. Poor solubility is detrimental to absorption after oral administration and can mask compound activity in bioassays in various ways, including underestimated activity, reduced hit rates in high-throughput screening (HTS), variable data outputs, inaccurate structure-activity relationship (SAR), and inaccurate in vitro absorption, distribution, metabolism, excretion and toxicity (ADMET) test results. The importance of solubility data in early stage drug profiling during development for prediction of oral absorption has also been widely discussed. Hence, solubility limitations should ideally be identified as early as possible prior to carrying out functional assays. Early evaluation of solubility in the drug discovery process is therefore of critical importance.

The solubility of a substance can be broadly defined as the maximum amount of the substance that dissolves in a specified volume of solvent. However, it is important to understand that the solubility of a compound can vary drastically depending on the condition of the solvent (e.g. temperature and pH) and the physiochemical properties of the compound (e.g. ionisation and crystallinity). These critical factors need to be considered during solubility determination in order to generate high quality solubility data that will be useful in the progression of compounds through the discovery and development stages. The kinetic solubility of a compound is the maximum solubility of the fastest precipitating species of the compound; this is often measured using a stock solution of the compound dissolved in an organic solvent, typically dimethyl sulfoxide (DMSO), as the starting material. Kinetic solubility values are strongly time- and method-dependent and hence are not expected to be reproducible between different laboratories using different protocols. The precipitate formed, which is rarely determined during the assessment, could be any combination of various possible solid states of the compound. Given the level of supersaturation that could occur when an organic solvent is diluted in water, kinetic solubility values are typically higher than the corresponding thermodynamic (equilibrium) solubility values. Solubility assays in the early discovery process often determine kinetic solubility. The lyophilisation solubility assay (LYSA) is a high throughput kinetic solubility assay for the early drug discovery process.

### Lyophilisation Solubility Assay (LYSA) Method

Samples were prepared in duplicate from 10 mM DMSO stock solution. After evaporation of DMSO with a centrifugal vacuum evaporator, the compounds were solved in 0.05 M phosphate buffer (pH 6.5), stirred for one hour and shaken for two hours. After one night, the solutions were filtered using a microtiter filter plate. Then the filtrate and its 1/10 dilution were analyzed by HPLC-UV. In addition a four-point calibration curve was prepared from the 10 mM stock solutions and used for the solubility determination of the compounds. The results were in μg/mL. In case the percentage of sample measured in solution after evaporation divided by the calculated maximum of sample amount was bigger than 80%, the solubility was reported as bigger than this value. It should be noted that metastable solid phases may form during the evaporation step, and the method might therefore produce higher solubility values than assays using the stable polymorph as the starting material.

The thermodynamic (equilibrium) solubility is the saturation solubility of a compound at the end of the dissolution process, where the dissolved compound is in equilibrium with the undissolved material in excess. The thermodynamic solubility is determined by dispensing a solid compound into a solvent. This is often deemed as the true solubility of the compound and is a critical piece of information in formulation development. The thermodynamic solubility assay (THESA) is a widely used tool for the measurement of equilibrium solubility for the later drug discovery process and is considered as a gold standard solubility assay.

### Thermodynamic Solubility Assay (THESA) Method

Approximately 1-2 mg of each compound was added into 300 μL of 50 mM phosphate buffer (pH 6.5) at room temperature (22±2° C.). Each sample is placed in a microanalysis tube, which was sonicated for 1 h and shaken for 2 h. All suspensions were left overnight (about 16 h). On the next day, the samples were filtered into a V-bottom plate with a micronic filterplate to separate the solid material from the solution. Then, all solutions were diluted into 96 well V-bottom microplates and analyzed by UPLC-UV. The calibration line was established by UPLC using 5 different dilutions of the compound in a DMSO stock solution, made in micronic tubes (concentration approximately 1 mg/mL). Dilutions took place with DMSO. From this regression equation the solubility of the compound was determined. In cases where the drug was completely dissolved in the buffer, the value for equilibrium solubility was assumed to be higher than the value determined by UPLC and reported as such.

The results clearly showed that both the kinetic solubility (LYSA) and thermodynamic (equilibrium) solubility (THESA) of the compounds of this invention were greatly improved compared to the reference compound RO14.

TABLE 2
LYSA and THESA of the compounds of this invention

| Example No. | LYSA (μg/mL) | THESA (μg/mL) |
|---|---|---|
| RO14 | 69 | 33 |
| Example 1 | >540 | 929 |
| Example 2 | >540 | 2778 |
| Example 3 | >600 | |
| Example 4 | >640 | |
| Example 5 | >570 | |
| Example 6 | >630 | |
| Example 7 | >540 | |
| Example 8 | >520 | |
| Example 9 | >650 | |
| Example 10 | >650 | |
| Example 11 | >620 | |
| Example 12 | >560 | |
| Example 13-A | >530 | 2379 |
| Example 13-B | >530 | |
| Example 14-A | 150 | |
| Example 14-B | 170 | |
| Example 15-A | >560 | 4059 |
| Example 15-B | >560 | >4967 |
| Example 16-A | >560 | 1902 |
| Example 16-B | >560 | 3453 |
| Example 17 | >540 | |
| Example 18-A | >580 | 3003 |
| Example 18-B | >580 | 4023 |
| Example 19 | >580 | 2366 |

### Example 22

### Dissolution Test: Fasted State Simulated Intestinal Fluid (FaSSIF) and Fed State Simulated Intestinal Fluid (FeSSIF)

Drug dissolution in the physiological environment of the gastrointestinal tract is the primary step in the oral absorption process from a pharmaceutical dosage form. Since only dissolved drug can permeate the mucosa at the absorptive sites in the gastrointestinal tract, both the solubility of the drug and its dissolution rate are crucial for its in vivo behavior. It is crucial to run dissolution tests under conditions that closely resemble the key parameters of human gastrointestinal physiology. In addition to the choice of adequate equipment and appropriate instrument parameters, the use of physiologically relevant dissolution media is of great importance.

Biorelevant gastrointestinal media that simulate the fasted and fed states have been developed to mimic the condition in vivo as closely as possible. These media have been used to examine the solubility and dissolution characteristics of several classes of drugs to assist in predicting in vivo absorption behavior. Biorelevant in vitro dissolution testing is useful for qualitative forecasting of formulation and food effects on the dissolution and availability of orally administered drugs. It has been observed that biorelevant media can provide a more accurate simulation of pharmacokinetic profiles than simulated gastric fluid or simulated intestinal fluid. The formulation and preparation instructions for the biorelevant media are detailed below.

### Fasted State Simulated Intestinal Fluid (FaSSIF)

### Step 1: Preparation of Blank FaSSIF

0.420 g of NaOH (pellets), 3.954 g of NaH₂PO₄ (monohydrate) and 6.186 g of NaCl were dissolved in about 0.9 L of purified water. The pH was adjusted to 6.5 with either 1 N NaOH or 1 N HCl. The total volume of the mixture was made up to 1.0 L with purified water at room temperature.

### Step 2: Preparation of FaSSIF

To about 50 mL of blank FaSSIF buffer was added 0.2240 g of SIF Powder Original (Biorelevant.com Ltd, Product code: FFF03, UK), the mixture was stirred until powder was completely dissolved. The total volume of the mixture was made up to 100 mL with blank FaSSIF buffer at room temperature.

### Fed State Simulated Intestinal Fluid (FeSSIF)

### Step 1: Preparation of Blank FeSSIF

4.040 g of NaOH (pellets), 8.650 g of glacial acetic acid and 11.874 g of NaCl were dissolved in about 0.9 L of purified water. The pH was adjusted to 5 with either 1 N NaOH or 1 N HCl. The total volume of the mixture was made up to 1.0 L with purified water at room temperature.

### Step 2: Preparation of FeSSIF

To about 50 mL of blank FeSSIF buffer was added 1.120 g of SIF Powder Original (Biorelevant.com Ltd, Product code: FFF03, UK), the mixture was stirred until powder was completely dissolved. The total volume of the mixture was made up to 100 mL with blank FeSSIF buffer at room temperature.

### Dissolution Test Method

Approximately 1-2 mg of each compound was added in excess into 300 NL of buffer at room temperature. Each sample is placed in a microanalysis glass tube, which was sonicated for 1 h and agitated for 2 h. All suspensions were left overnight (about 16 h). On the next day, all pH values were measured with a pH-meter and the samples were filtered into a V-bottom plate with a micronic filterplate to separate the solid material from the solution. Then, all solutions were diluted into 96 well V-bottom microplates and analyzed by UPLC-UV.

The calibration line was established by UPLC using 5 different dilutions of the compound in a DMSO stock solution, made in micronic tubes (concentration approximately 1 mg/mL). Dilutions took place with DMSO (in special circumstances, with acetonitrile, or 80% HCl 0.1 N/20% acetonitrile). From this regression equation the solubility of the compound was determined. In cases where the compound was completely dissolved in the buffer, the value for equilibrium solubility was assumed to be higher than the value determined by UPLC and reported as such.

The results clearly showed that both the dissolution in the fasted state simulated intestinal fluid (FaSSIF) and in the fed state simulated intestinal fluid (FeSSIF) of the compounds of this invention were greatly improved compared to the reference compound RO14. Table 3. FaSSIF and FeSSIF of the compounds of this invention.

| Example No. | FaSSIF (μg/mL) | FeSSIF (μg/mL) |
|---|---|---|
| RO14 | 14 | 21 |
| Example 1 | 2527 | 3077 |
| Example 2 | 3519 | 4801 |
| Example 13-A | 2368 | 4672 |
| Example 15-A | 3074 | 3987 |
| Example 15-B | >3733 | >4267 |
| Example 16-A | 1799 | 2371 |
| Example 16-B | 784 | 1464 |
| Example 18-A | 3436 | 4536 |
| Example 18-B | >3767 | >5000 |

1. A compound of formula (I), 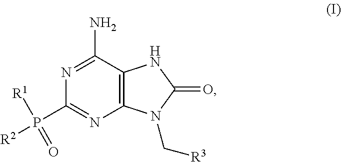
wherein
R¹ is C₁₋₆alkyl;
R² is C₁₋₆alkyl;
R³ is pyridinyl substituted by ((C₁₋₆alkyl)₂amino)C₁₋₆alkoxy, ((C₁₋₆alkyl)₂amino)C₁₋₆alkylamino, ((C₁₋₆alkyl)₂amino)C₁₋₆alkylpyrrolidinyl, (C₁₋₆alkyl)₂piperazinyl, (C₁₋₆alkylamino)C₁₋₆alkylamino, (C₁₋₆alkylpyrrolidinyl)amino, 2,3,3a,4,6,6a-hexahydro-1H-pyrrolo[3,4-c]pyrrolyl, 2,5-diazabicyclo[2.2.2]octanyl, aminopyrrolidinyl, C₁₋₆alkyl-1-oxa-4,9-diazaspiro[5.5]undecanyl, C₁₋₆alkylpiperazinyl, C₁₋₆alkylsulfonylpiperazinyl, pyrrolidinylC₁₋₆alkylamino or tetrahydropyranylamino;
or a pharmaceutically acceptable salt thereof.

2. A compound according to claim 1, wherein R¹ is methyl or ethyl.

3. A compound according to claim 1, wherein R¹ is methyl.

4. A compound according to claim 1, wherein R² is methyl, ethyl or propyl.

5. A compound according to claim 1, wherein R³ is 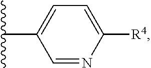
wherein R⁴ is ((C₁₋₆alkyl)₂amino)C₁₋₆alkoxy, ((C₁₋₆alkyl)₂amino)C₁₋₆alkylamino, ((C₁₋₆alkyl)₂amino)C₁₋₆alkylpyrrolidinyl, (C₁₋₆alkyl)₂piperazinyl, (C₁₋₆alkylamino)C₁₋₆alkylamino, (C₁₋₆alkylpyrrolidinyl)amino, 2,3,3a,4,6,6a-hexahydro-1H-pyrrolo[3,4-c]pyrrolyl, 2,5-diazabicyclo[2.2.2]octanyl, aminopyrrolidinyl, C₁₋₆alkyl-1-oxa-4,9-diazaspiro[5.5]undecanyl, C₁₋₆alkylpiperazinyl, C₁₋₆alkylsulfonylpiperazinyl, pyrrolidinylC₁₋₆alkylamino or tetrahydropyranylamino.

6. A compound according to claim 1, wherein R³ is 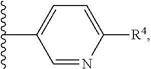
wherein R⁴ is ((C₁₋₆alkyl)₂amino)C₁₋₆alkylamino or (C₁₋₆alkylamino)C₁₋₆alkylamino.

7. A compound according to claim 1, wherein R³ is 2-(dimethylamino)ethylamino or 2-(methylamino)ethylamino.

8. A compound according to claim 1, wherein
R¹ is C₁₋₆alkyl;
R² is C₁₋₆alkyl;
R³ is 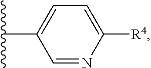
wherein R⁴ is ((C₁₋₆alkyl)₂amino)C₁₋₆alkylamino or (C₁₋₆alkylamino)C₁₋₆alkylamino;
or a pharmaceutically acceptable salt thereof.

9. A compound according to claim 8, wherein
R¹ is methyl;
R² is methyl, ethyl or propyl;
R³ is 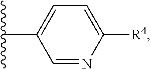
wherein R⁴ is 2-(dimethylamino)ethylamino or 2-(methylamino)ethylamino;
or a pharmaceutically acceptable salt thereof.

10. A compound selected from:
6-amino-9-[[6-[2-(dimethylamino)ethylamino]-3-pyridyl]methyl]-2-dimethylphosphoryl-7H-purin-8-one;
6-amino-9-[[6-[2-(dimethylamino)ethoxy]-3-pyridyl]methyl]-2-dimethylphosphoryl-7H-purin-8-one;
6-amino-9-[[6-(3-aminopyrrolidin-1-yl)-3-pyridyl]methyl]-2-dimethylphosphoryl-7H-purin-8-one;
6-amino-2-dimethylphosphoryl-9-[[6-(3,3-dimethylpiperazin-1-yl)-3-pyridyl]methyl]-7H-purin-8-one;
6-amino-9-[[6-(2,5-diazabicyclo[2.2.2]octan-2-yl)-3-pyridyl]methyl]-2-dimethylphosphoryl-7H-purin-8-one;
9-[[6-(2,3,3a,4,6,6a-hexahydro-1H-pyrrolo[3,4-c]pyrrol-5-yl)-3-pyridyl]methyl]-6-amino-2-dimethylphosphoryl-7H-purin-8-one;
6-amino-2-dimethylphosphoryl-9-[[6-(2-pyrrolidin-1-ylethylamino)-3-pyridyl]methyl]-7H-purin-8-one;
6-amino-2-dimethylphosphoryl-9-[[6-(tetrahydropyran-4-ylamino)-3-pyridyl]methyl]-7H-purin-8-one;
6-amino-9-[[6-[3-[(dimethylamino)methyl]pyrrolidin-1-yl]-3-pyridyl]methyl]-2-dimethylphosphoryl-7H-purin-8-one;
6-amino-2-dimethylphosphoryl-9-[[6-(4-methyl-1-oxa-4,9-diazaspiro[5.5]undecan-9-yl)-3-pyridyl]methyl]-7H-purin-8-one;
6-amino-2-dimethylphosphoryl-9-[[6-[(1-methylpyrrolidin-3-yl)amino]-3-pyridyl]methyl]-7H-purin-8-one;
6-amino-2-dimethylphosphoryl-9-[[6-[2-[ethyl(methyl)amino]ethylamino]-3-pyridyl]methyl]-7H-purin-8-one;
6-amino-2-[(R)-ethyl(methyl)phosphoryl]-9-[[6-(4-methylpiperazin-1-yl)-3-pyridyl]methyl]-7H-purin-8-one;
6-amino-2-[(S)-ethyl(methyl)phosphoryl]-9-[[6-(4-methylpiperazin-1-yl)-3-pyridyl]methyl]-7H-purin-8-one;
6-amino-2-[(R)-ethyl(methyl)phosphoryl]-9-[[6-(4-methylsulfonylpiperazin-1-yl)-3-pyridyl]methyl]-7H-purin-8-one;
6-amino-2-[(S)-ethyl(methyl)phosphoryl]-9-[[6-(4-methylsulfonylpiperazin-1-yl)-3-pyridyl]methyl]-7H-purin-8-one;
6-amino-9-[[6-[2-(dimethylamino)ethylamino]-3-pyridyl]methyl]-2-[(R)-ethyl(methyl)phosphoryl]-7H-purin-8-one;
6-amino-9-[[6-[2-(dimethylamino)ethylamino]-3-pyridyl]methyl]-2-[(S)-ethyl(methyl)phosphoryl]-7H-purin-8-one;
6-amino-9-[[6-[2-(dimethylamino)ethoxy]-3-pyridyl]methyl]-2-[(R)-ethyl(methyl)phosphoryl]-7H-purin-8-one;
6-amino-9-[[6-[2-(dimethylamino)ethoxy]-3-pyridyl]methyl]-2-[(S)-ethyl(methyl)phosphoryl]-7H-purin-8-one;
6-amino-2-[(R)-ethyl(methyl)phosphoryl]-9-[[6-[2-(methylamino)ethylamino]-3-pyridyl]methyl]-7H-purin-8-one;
6-amino-9-[[6-[2-(dimethylamino)ethylamino]-3-pyridyl]methyl]-2-[(S)-methyl(propyl)phosphoryl]-7H-purin-8-one;
6-amino-9-[[6-[2-(dimethylamino)ethylamino]-3-pyridyl]methyl]-2-[(R)-methyl(propyl)phosphoryl]-7H-purin-8-one; and
6-amino-2-diethylphosphoryl-9-[[6-[2-(dimethylamino)ethylamino]-3-pyridyl]methyl]-7H-purin-8-one;
or a pharmaceutically acceptable salt thereof.

11. A process for the preparation of a compound according to claim 1 comprising the following step:
a) reacting a compound of formula (III), 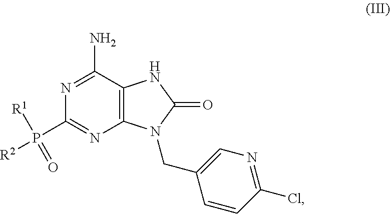
and an amine or alcohol HR⁴ (II), in a neat reaction; or in the presence of inorganic base, wherein the base is sodium hydride; or in the presence of organometallic catalyst system, wherein the catalyst system is selected from Pd₂(dba)₃/RuPhos/t-BuONa, Pd₂(dba)₃/BrettPhos/t-BuONa, and Pd-PEPPSI-IPentCl/t-BuOK; wherein R¹, R², and R⁴ are defined as in Formula (I).

12. A compound of formula (I) or a pharmaceutically acceptable salt thereof manufactured according to the process of claim 11.

13. A pharmaceutical composition comprising a compound according to claim 1 and a pharmaceutically acceptable excipient.

14. A compound or pharmaceutically acceptable salt according to claim 1 for use as therapeutically active substance.

15. (canceled)

16. A method of boosting innate immune response in myeloid cells that express TLR7, comprising contacting the cells with a compound according to claim 1 as an agonist of TLR7.

17-19. (canceled)

20. A method for the treatment of cancer in a patient in need thereof, wherein the cancer is selected from pancreatic ductal adenocarcinoma, colorectal carcinoma, melanoma, hepatocellular carcinoma, cholangiocarcinoma, breast carcinoma, cervical carcinoma, endometrial carcinoma, ovarian carcinoma, head and neck squamous cell carcinoma, adenoid cystic carcinoma, extensive-stage small cell lung carcinoma, non-small cell lung carcinoma, muscle-invasive bladder carcinoma, nodular basal cell carcinoma and squamous cell carcinoma, which method comprises administering to the patient a therapeutically effective amount of a compound as defined in claim 1.

21. (canceled)

22. The method of claim 20, wherein the cancer is selected from pancreatic ductal adenocarcinoma and colorectal carcinoma.
